(19) United States
(12) Patent Application Publication
(10) Pub. No.: US 2026/0347827 A1
(43) Pub. Date: Nov. 5, 2026

(54) METHOD FOR CONTROLLING CRISPR-CAS9 GENOME EDITING VIA MATRIX MECHANICAL PROPERTIES

(71) Applicant: The Johns Hopkins University, Baltimore, MD (US)

(72) Inventors: Luo Gu, Baltimore, MD (US); Joon Hyuck Eoh, Baltimore, MD (US)

(21) Appl. No.: 19/485,755

(22) PCT Filed: May 20, 2024
(86) PCT No.: PCT/US2024/030230

§ 371 Date: Nov. 19, 2025

Related U.S. Application Data
(60) Provisional application No. 63/503,308, filed on May 19, 2023.

(51) Int. Cl. C12N 15/10; A61K 48/00; C08J 3/075; C08L 33/26; C12N 9/22; C12N 15/90

(52) U.S. Cl. CPC C12N 15/10; A61K 48/0008; C08J 3/075; C08L 33/26; C12N 9/226; C12N 15/907; C08J 2333/26; C08J 2389/00; C12N 2310/20

(57) ABSTRACT

Hydrogels, such as a polyacrylamide (PAM) hydrogel, so constructed to have a Young's modulus of a particular value, or an alginate hydrogel, crosslinked with calcium ions are disclosed, such hydrogels having a varying range of stiffnesses and/or viscoelasticity. The hydrogels may comprise one or more extracellular matrix (ECM) proteins bound to a surface thereof, or comprise one or more seeded cells, wherein such cells may comprise a Cas9 cargo. These hydrogels are prepared for CRISPR-Cas9 genome editing, utilized for viral delivery, including delivery with adeno-associated virus (AAV) or lentivirus vectors.

### CROSS-REFERENCE TO RELATED APPLICATIONS

This application claims benefit of U.S. Provisional Patent Application No. 63/503,308 filed on May 19, 2023, which is incorporated herein by reference in its entirety.

### BACKGROUND

CRISPR-Cas9 gene editing has generated enormous impact in the overall scientific community and opened countless new avenues of research. This impact is due, in part, to its ease of use as the system in its simplest form requires only the Cas9 endonuclease, single guide RNA (sgRNA), and a donor template, if required. Eoh and Gu, 2019. To actualize the full potential of the CRISPR-Cas9 system, however, there is a need to improve the delivery and editing processes. Several barriers, however, to improving these processes exist.

### FIELD

Hydrogels having a varying range of stiffnesses and/or viscoelasticity and their use in enhancing gene editing are disclosed.

### SUMMARY

The presently disclosed subject matter provides hydrogels having a varying range of stiffnesses and/or viscoelasticity and their use in enhancing gene editing. In some aspects, the hydrogel comprises a hydrogel for CRISPR-Cas9 genome editing, wherein the hydrogel has a Young's modulus between about 0 . . . . In particular aspects, the Young's modulus is selected from about 0.2 kPa, 2 kPa, 2.5 kPa, 4 kPa, 10 kPa, 20 kPa, 30 kPa, 50 kPa, 100 kPa, and 150 kPa.

In certain aspects, the hydrogel is selected from a polyacrylamide (PAM) hydrogel, an alginate, a collagen, and polyethylene glycol (PEG).

In particular aspects, the hydrogel comprises a PAM hydrogel. In more particular aspects, the PAM hydrogel comprises between about 2% wt % (w/v) to about 20% wt % (w/v) acrylamide and between about 0.010 wt % (w/v) to about 0.5 wt % (w/v) of bis-acrylamide. In certain aspects, the PAM hydrogel further comprises one or more of ammonium persulfate, tetramethylethylenediamine (TEMED), phosphate-buffered saline (PBS), and water. In more certain aspects, the PAM hydrogel comprises a 1:100 total volume of ammonium sulfate and a 1:1000 total volume of tetramethylethylenediamine.

In other aspects the hydrogel comprises an alginate hydrogel. In certain aspects, the alginate hydrogel is crosslinked with calcium ions.

In other aspects, the hydrogel comprises polyethylene glycol (PEG).

In certain aspects, the hydrogel further comprises one or more extracellular matrix (ECM) proteins bound to a surface thereof. In particular aspects, the one or more ECM proteins are selected from collagen I, laminin, RGD-containing peptides, and fibronectin.

In certain aspects, the hydrogel comprises one or more seeded cells. In particular aspects, the one or more cells is selected from U2OS.EGFP cells, human mesenchymal stem cells (MSCs), T cells, hematopoietic stem/progenitor cells (HSCs), neural stem/progenitor cells, human dermal fibroblasts, macrophages, and induced pluripotent stem cells (iPSCs).

In certain aspects, the one or more cells comprise an RNA-guided nuclease and a guide RNA. In certain aspects, the one or more cells comprise a Cas9 cargo. In particular aspects, the Cas9 cargo is selected from a plasmid DNA, an mRNA, and a protein. In certain aspects, the plasmid DNA comprises a sequence encoding a Cas9 nuclease and a sequence encoding a single guide RNA (sgRNA). In certain aspects, the one or more cells comprise a Cas9 mRNA and a guide RNA. In certain aspects, the Cas9 protein is a ribonucleoprotein (RNP) complex comprising a Cas protein and a guide RNA. In more particular aspects, the Cas9 cargo is selected from a mCherry-Cas9 plasmid, a Cas9 plasmid, a Cas9 mRNA, Cas9 ribonucleoprotein (RNP), or other forms of Cas9 including, but limited to, saCas9, dCas9, FokI-Fused dCas9, eSpCas9, xCas9, SpRY/SpG, HypaCas9, and High-Fidelity Cas9.

In other aspects, the presently disclosed subject matter provides a method for preparing a hydrogel for CRISPR-Cas9 genome editing, the method comprising: (a) providing a hydrogel having a Young's modulus between about 0.1 kPa and about 200 kPa; (b) functionalizing the hydrogel with one or more ECM proteins to form a functionalized hydrogel; and (c) seeding the functionalized hydrogel with one or more cells.

In certain aspects, the method further comprises incubating the one or more cells for a period of time. In certain aspects, the method further comprises transfecting the seeded cells with a Cas9 cargo. In certain aspects, the method further comprises transfecting the seeded cells with a Cas9 mRNA and a guide RNA. In particular aspects, the transfecting comprises a transfection system selected from polyethylenimine (PEI), a lipid, a liposome, and electroporation. In certain aspects, the lipid or liposome comprises one or more components selected from 2,3-dioleoyloxy-N-[2 (sperminecarboxamido)ethyl]-N,N-dimethyl-1-propaniminium trifluoroacetate (DOSPA), dioleoylphosphatidylethanolamine (DOPE), cholesterol, 2-dioleoyl-3-trimethylammonium-propane (DOTAP), 1,2-distearoyl-sn-glycero-3-phosphocholine (DSPC), 1,2-dioleoyl-sn-glycero-3-phosphocholine (DOPC), polyethylene glycol (PEG), and combinations thereof. In particular aspects, the transfection system comprises linear or branched PEI.

In certain aspects, the method further comprises viral delivery, including delivery with adeno-associated virus (AAV) or lentivirus vectors.

In certain aspects, the presently disclosed subject matter provides a method for improving DNA editing of CRISPR-Cas9, the method comprising: providing a hydrogel having a Young's modulus between about 2 kPa and about 200 kPa; seeding the hydrogel with one or more cells; and transfecting the seeded cells with a Cas9 cargo; wherein DNA editing efficiency is enhanced. In certain aspects, cytoskeletal alignment, cytoskeletal tension, or chromatin accessibility of the seeded cells is increased. In certain aspects, the hydrogel is a hydrogel described herein. In certain aspects, seeding the hydrogel with one or more cells is a seeding method described herein. In certain aspects, the cells are selected from T-cells, human mesenchymal stem cells (MSCs), hematopoietic stem/progenitor cells (HSCs), neural stem/progenitor cells, human dermal fibroblasts, macrophages, and induced pluripotent stem cells (iPSCs). In certain aspects, transfecting the seeded cells with a Cas9 cargo is a transfection method described herein. In certain aspects, homology-directed repair (HDR) efficiency is enhanced as compared to HDR efficiency of Cas9 cargo transfection of seeded cells on tissue culture plastic (TCP). In certain aspects, the enhanced DNA editing efficiency is the enhanced DNA editing efficiency as measured or described herein, e.g., bp insertions or deletions. In certain aspects, the enhanced DNA editing efficiency is the enhanced DNA editing efficiency as compared to DNA editing efficiency of Cas9 cargo transfection of seeded cells on tissue culture plastic (TCP).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1A, FIG. 1B, FIG. 1C, FIG. 1D, FIG. 1E, FIG. 1F, FIG. 1G, and FIG. 1H show that matrix stiffness enhances gene editing in U2OS.EGFP cells as demonstrated with multiple transfection methods and hydrogel systems. (FIG. 1A) Experimental setup for hydrogel-based transfection studies for chemical transfection, as well as electroporation. (i) For chemical transfection, cells are first seeded onto hydrogels of varying stiffnesses and allowed to adhere overnight. All samples were then transfected with polyethylenimine (PEI) or Lipofectamine™ 2000. (ii) The Cas9 cargo was delivered via electroporation after which cells were immediately seeded onto hydrogels. As a control, all experiments were performed in parallel on a tissue culture polystyrene (TCPS) coverslip mounted on a glass slide to better mimic the physical dimensions of hydrogels. (FIG. 1B) Representative images of enhanced green fluorescence protein (EGFP) gene editing in U2OS.EGFP cells cultured on polyacrylamide (PAM) hydrogels of varying stiffnesses and TCPS. Gene editing on PAM hydrogels of varying stiffnesses demonstrates significantly improved editing efficiencies over TCPS and an increase in gene editing with stiffness. This improvement is demonstrated with three separate transfection platforms: (FIG. 1C) Linear PEI, (FIG. 1D) Lipofectamine™ 2000, and (FIG. 1E) Electroporation. This increasing trend in editing with matrix stiffness also is demonstrated on three other hydrogel systems: (FIG. 1F) Alginate, (FIG. 1G) Collagen, and (FIG. 1H) PEG. Scale bars=25 μm. Data are presented as mean±SEM with at least n=3 replicates. Significance levels were set at: *P<0.05; **P<0.01; and ***P<0.001, while #denotes a statistically significant difference from the TCPS control. Significance levels set at: #P<0.05; ##P<0.01; and ###P<0.001;

FIG. 2A and FIG. 2B demonstrate that Cas9 expression and gene editing kinetics in cells are improved when cultured on hydrogels. (FIG. 2A) All samples were transfected with a mCherry-Cas9 plasmid and the expression was measured at (i) 12 hours, (ii) 24 hours, and (iii) 48 hours via image analysis. (FIG. 2B) Flow cytometry studies also were performed at (i) 24 hours, (ii) 48 hours, and (iii) 72 hours to measure the gene editing kinetics. Data are presented as mean±SEM with at least n=3 replicates. Significance levels were set at: *P<0.05; **P<0.01; and ***P<0.001, while #denotes a statistically significant difference from the TCPS control. Significance levels set at: #P<0.05; ##P<0.01; and ###P<0.001;

FIG. 3A, FIG. 3B, FIG. 3C, and FIG. 3D demonstrate that substrate stiffness improves Cas9 editing efficiency for other forms of Cas9, as well as in multiple cell types and gene targets. (FIG. 3A) Flow cytometry data for editing efficiency for studies using Cas9 mRNA and (FIG. 3B) Cas9 RNP. (FIG. 3C) Sequencing data for AAVS1 gene editing in U2OS.EGFP cells. The nature of the edits are presented for the (i) TCPS, (ii) 2 kPa, (iii) 20 kPa, and (iv) 100 kPa conditions, as well as the (v) overall editing efficiency values. (FIG. 3D) Sequencing data for AAVS1 gene editing in human mesenchymal stem cells. The nature of the edits are presented for the (i) TCPS, (ii) 1 kPa, (iii) 2 kPa, (iv) 20 kPa, and (v) 100 kPa conditions as well as the (vi) overall editing efficiency values. Data are presented as mean±SEM with at least n=3 replicates. Significance levels were set at: *P<0.05; **P<0.01; and ***P<0.001, while #denotes a statistically significant difference from the TCPS control. Significance levels set at: #P<0.05; ##P<0.01; and ###P<0.001;

FIG. 4A, FIG. 4B, FIG. 4C, FIG. 4D, FIG. 4E, FIG. 4F, FIG. 4G, and FIG. 4H demonstrate that alteration of the cytoskeleton, nucleoskeleton, and mechanotransduction pathways directly impacts the substrate-induced effects of gene editing. Gene editing efficiency was assessed via flow cytometry after treatment with (FIG. 4A) myosin light chain kinase inhibitor, ML7, (FIG. 4B) actin polymerization inhibitor, cytochalasin-D, (FIG. 4C) Rho kinase inhibitor Y-27632, (FIG. 4D) Rho GTPase activator, lysophosphatidic acid, (FIG. 4E) YAP inhibitor, verteporfin, (FIG. 4F) Nesprin-1 siRNA (FIG. 4G) SUNI siRNA, and (FIG. 4H) Lamin-A/C siRNA. Data are presented as mean±SEM with at least n=3 replicates. Significance levels were set at: *P<0.05; **P<0.01; and ***P<0.001. For the small molecule inhibitors, * indicates significance compared to the positive control condition. For siRNA studies, * indicates significance in comparison to the NTC-siRNA condition;

FIG. 5A, FIG. 5B, FIG. 5C, FIG. 5D, FIG. 5E, and FIG. 5F illustrate the proposed mechanism for stiffness-enhanced gene editing. (FIG. 5A) Schematic of the delivery and editing process for CRISPR-Cas9. The stages of Cas9 delivery and editing can be divided into the following phases: (1) Uptake; (2) Nuclear transport of the plasmid Cas9; (3) Transcription and nuclear export; (4) Translation and RNP assembly; and finally, (5) Nuclear shuttling and genome editing. By investigating various stages of the delivery process, we can potentially determine one(s) in which mechanotransduction has the greatest impact and continue developing a potential mechanism. (FIG. 5B) Lamin-A/C and (FIG. 5C) SUNI siRNA knockdown studies were performed to determine if alteration of the LINC complex would directly impact Cas9 protein transport to the nucleus and subsequent editing. (FIG. 5D) After electroporation with Cas9-RFP, cell nuclei were extracted and RFP expression was measured via flow cytometry at (i) 6 hours, (ii) 9 hours, (iii) 12 hours, (iv) 15 hours and (v) 22 hours. (FIG. 5E) Chromatin accessibility of the AAVS1 gene was quantified by qPCR. (FIG. 5F) A schematic that summarizes the potential mechanism on how stiffness enhances gene editing. Substrate stiffness enhances CRISPR gene editing through two mechanisms: (i) Increased cytoskeletal involvement through hydrogel culture leads to quicker nucleocytoplasmic shuttling. (ii) Cytoskeletal tension leads to increased force transmission to the nucleus via the LINC complex, and underlying nucleoskeleton. This results in increased chromatin accessibility to the Cas9 RNP. These processes combine resulting in editing efficiencies higher than those obtained on TCPS and increasing in a stiffness-dependent manner. Data are presented as mean±SEM with at least n=3 replicates. Significance levels were set at: *P<0.05; **P<0.01; and ***P<0.001. For the nuclear transport study, * indicates significance compared to the TCPS condition. For siRNA studies, * indicates significance in comparison to the NTC-siRNA condition;

FIG. 6 demonstrates cell spreading on hydrogels presented as the projected cell area;

FIG. 7 shows genomic DNA measurements from samples obtained at the end of a transfection;

FIG. 8 shows nucleus height measurements taken from TCPS and hydrogels as an indicator of nuclear flattening;

FIG. 9 shows Western blot images of the LINC complex and Lamin A/C;

FIG. 10 shows plasmid uptake data obtained at 4 hours, 12 hours, and 22 hours;

FIG. 11 shows nuclear flow cytometry data measuring the Cy5-labeled plasmid content in the nucleus;

FIG. 12 shows nanoparticle retention measurements performed on the hydrogels with and without rinsing with culture media. Data are presented as mean±SEM with at least n=3 replicates. Significance levels were set at: *P<0.05; **P<0.01; and ***P<0.001;

FIG. 13 shows editing efficiency as a function of viscoelasticity;

FIG. 14 shows substrate stiffness improves CRISPR-Cas9 editing efficiency in homology-directed repair (HDR) pathway. This bar graph illustrates the percentage of cells experiencing HDR events cultured on varying substrate stiffness conditions. * denotes a statistically significant difference from the TCPS control. Significance levels set at: *P<0.05; **P<0.01; and ***P<0.001; and

FIG. 15 shows substrate stiffness improves CRISPR-Cas9 editing efficiency in T cells. Electroporation of CD3⁺ T cells with the Cas9 RNP and AAVS1 sgRNA resulted in higher editing efficiencies when using hydrogel substrates with 20 and 100 kPa conditions compared to the conventional tissue culture plastic (TCPS) condition.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The presently disclosed subject matter provides methods for controlling CRISPR-Cas9 genome editing by tuning the mechanical properties of the extracellular matrix. More particularly, the substrate rigidity and/or viscoelasticity are used to achieve this purpose.

With regard to substrate rigidity, in some embodiments, polyacrylamide (PAM) hydrogels with varying cross-linker concentrations are used to generate hydrogels with varying stiffnesses. The gels are then coated with cell adhesion ligands to promote cell attachment.

For the tuning of viscoelasticity, in some embodiments, RGD peptide-modified alginates of varying molecular weights are used to form hydrogels via ionic crosslinking from calcium ions, e.g., calcium sulfate particles. The different molecular weights correspond to the varying levels of viscoelasticity while the stiffness also can be tuned by adjusting the calcium concentrations.

After cells are seeded on the gels and allowed to adhere, e.g., overnight, samples are transfected with the Cas9 cargo using a transfection system. In certain embodiments, the method further comprises transfecting the seeded cells with a Cas9 mRNA and a guide RNA. The gene editing efficiency can be using methods known in the art, such as flow cytometry or a commercial gene cleavage detection assay.

The presently disclosed subject matter demonstrates that culturing and transfecting cells on ECM-coated PAM gels results in higher editing efficiency using less transfection reagents compared to parallel studies on conventional tissue culture methods. Without wishing to be bound to any one particular theory, it is thought that this improvement is due to enhanced cell-substrate interactions between the cells and gels, which leads to a greater uptake of nanoparticles, a quicker onset of gene editing, and an overall improved gene editing efficiency. Because less of the transfection reagents are required, this strategy could prove useful for hard-to-transfect and hard-to-edit cell lines. Further, this technology can easily be scaled up and holds promise for potential cell therapy applications.

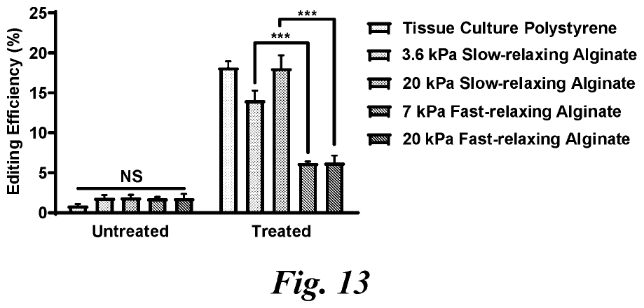

In regard to cells cultured and transfected on the viscoelastic alginate substrates, editing efficiencies are significantly lower than those observed in standard tissue culture methods. Further, higher amounts of transfection reagents are required to observe detectable levels of genome editing. Thus, the presently disclosed data demonstrate that the viscoelasticity plays a major role in gene editing. This finding could be a potential explanation for the rather low levels of genome editing detected found in vivo where many tissues exhibit viscoelastic properties. Because of this observation, culturing cells on viscoelastic substrates could serve as a useful and powerful tool for in vitro optimization of drug delivery systems or as a tool to further elucidate the impact of tissue viscoelasticity on drug delivery. See, for example, FIG. 13, which demonstrates editing efficiency as a function of elasticity for 3.6 kPa slow-relaxing alginate, 20 kPa slow-relaxing alginate, 7 kPa fast-relaxing alginate, and 20 kPa fast-relaxing alginate.

More particularly, in some embodiments, the hydrogel comprises a hydrogel for CRISPR-Cas9 genome editing, wherein the hydrogel has a Young's modulus between about 0.1 kPa and about 200 kPa, including about 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 kPa. In particular embodiments, the Young's modulus is selected from 0.2 kPa, 2 kPa, 2.5 kPa, 4 kPa, 10 kPa, 20 kPa, 30 kPa, 50 kPa, 100 kPa, and 150 kPa.

In certain embodiments, the hydrogel is selected from a polyacrylamide (PAM) hydrogel, an alginate, a collagen, and polyethylene glycol (PEG).

In particular embodiments, the hydrogel comprises a PAM hydrogel. In more particular embodiments, the PAM hydrogel comprises between about 2% wt % (w/v) to about 20% wt % (w/v) acrylamide, including between about 2, 3, 4, 5, 6, 7, 8, 9, 10, 11, 12, 13, 14, 15, 16, 17, 18, 19, and 20 wt % (w/v) acrylamide and between about 0.010 wt % (w/v) to about 0.5 wt % (w/v) of bis-acrylamide, including between about 0.01, 0.02, 0.05, 0.1, 0.15, 0.2, 0.25, 0.3, 0.35, 0.4, 0.45, and 0.5 wt % (w/v) bis-acrylamide. One of ordinary skill in the art would recognize that other methods can be used to initiate the crosslinking reaction including, but not limited to, ammonium persulfate and tetramethylethylenediamine (APS/TEMED) and UV crosslinking.

In certain embodiments, the PAM hydrogel further comprises one or more of ammonium persulfate, tetramethylethylenediamine (TEMED), phosphate-buffered saline (PBS), and water. In more certain embodiments, the PAM hydrogel comprises a 1:100 total volume of ammonium sulfate and a 1:1000 total volume of tetramethylethylenediamine.

In other embodiments the hydrogel comprises an alginate hydrogel. In certain embodiments, the alginate hydrogel is crosslinked with calcium sulfate.

In other embodiments, the hydrogel comprises polyethylene glycol (PEG).

In certain embodiments, crosslinking is accomplished through UV-mediated free radical crosslinking.

In certain embodiments, the hydrogel further comprises one or more extracellular matrix (ECM) proteins bound to a surface thereof. In particular embodiments, the one or more ECM proteins are selected from collagen I, laminin, RGD-containing peptides, and fibronectin.

In certain embodiments, the hydrogel comprises one or more seeded cells. In particular embodiments, the one or more cells is selected from U2OS.EGFP cells, human mesenchymal stem cells (MSCs), T cells, hematopoietic stem/progenitor cells (HSCs), neural stem/progenitor cells, human dermal fibroblasts, macrophages, and induced pluripotent stem cells (iPSCs).

In certain embodiments, the one or more cells comprise an RNA-guided nuclease and a guide RNA. In certain embodiments, the one or more cells further comprise a Cas9 cargo In some embodiments, the one or more cells further comprise a Cas9 cargo. In some embodiments, the one or more cells further comprises a single guide RNA (sgRNA). In particular embodiments, the Cas9 cargo is selected from a plasmid DNA, an mRNA, and a protein. In certain embodiments, the plasmid DNA comprises a sequence encoding a Cas9 nuclease and a sequence encoding a single guide RNA (sgRNA). In certain embodiments, the one or more cells comprise a Cas9 mRNA and a guide RNA. In certain embodiments, the Cas9 protein is a ribonucleoprotein (RNP) complex comprising a Cas protein and a guide RNA. In more particular embodiments, the Cas9 cargo is selected from a Cas9 plasmid, including a mCherry-Cas9 plasmid, a Cas9 mRNA, Cas9 ribonucleoprotein (RNP), or other forms of Cas9 including, but limited to, saCas9, dCas9, FokI-Fused dCas9, eSpCas9, xCas9, SpRY/SpG, HypaCas9, High-Fidelity Cas9.

In other embodiments, the presently disclosed subject matter provides a method for preparing a hydrogel for CRISPR-Cas9 genome editing, the method comprising: (a) providing a hydrogel having a Young's modulus between about 0.1 kPa and about 200 kPa; (b) functionalizing the hydrogel with one or more ECM proteins to form a functionalized hydrogel; and (c) seeding the functionalized hydrogel with one or more cells.

In certain embodiments, the method further comprises incubating the one or more cells for a period of time. In certain embodiments, the method further comprises transfecting the seeded cells with a Cas9 cargo and/or a single guide RNA (sgRNA). In particular embodiments, the transfecting comprises a transfection system selected from polyethylenimine (PEI), a lipid, a liposome, and electroporation. In certain embodiments, the lipid or liposome comprises one or more components selected from 2,3-dioleoyloxy-N-[2 (sperminecarboxamido)ethyl]-N,N-dimethyl-1-propaniminium trifluoroacetate (DOSPA), dioleoylphosphatidylethanolamine (DOPE), cholesterol, 2-dioleoyl-3-trimethylammonium-propane (DOTAP), 1,2-distearoyl-sn-glycero-3-phosphocholine (DSPC), 1,2-dioleoyl-sn-glycero-3-phosphocholine (DOPC), polyethylene glycol (PEG), and combinations thereof. In particular embodiments, the transfection system comprises linear or branched PEI.

In certain embodiments, the method further comprises viral delivery, including delivery with adeno-associated virus (AAV) or lentivirus vectors.

In certain embodiments, the presently disclosed subject matter provides a method for improving DNA editing of CRISPR-Cas9, the method comprising: providing a hydrogel having a Young's modulus between about 2 kPa and about 200 kPa; seeding the hydrogel with one or more cells; and transfecting the seeded cells with a Cas9 cargo; wherein DNA editing efficiency is enhanced. In certain embodiments, cytoskeletal alignment, cytoskeletal tension, or chromatin accessibility of the seeded cells is increased. In certain embodiments, the hydrogel is a hydrogel described herein. In certain embodiments, seeding the hydrogel with one or more cells is a seeding method described herein. In certain embodiments, the cells are selected from T-cells, human mesenchymal stem cells (MSCs), hematopoietic stem/progenitor cells (HSCs), neural stem/progenitor cells, human dermal fibroblasts, macrophages, and induced pluripotent stem cells (iPSCs). In certain embodiments, transfecting the seeded cells with a Cas9 cargo is a transfection method described herein. In certain embodiments, homology-directed repair (HDR) efficiency is enhanced as compared to HDR efficiency of Cas9 cargo transfection of seeded cells on tissue culture plastic (TCP). In certain embodiments, the enhanced DNA editing efficiency is the enhanced DNA editing efficiency as measured or described herein, e.g., bp insertions or deletions. In certain embodiments, the enhanced DNA editing efficiency is the enhanced DNA editing efficiency as compared to DNA editing efficiency of Cas9 cargo transfection of seeded cells on tissue culture plastic (TCP).

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments±100%, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Method for Controlling CRISPR-Cas9 Genome Editing Via Matrix Mechanical Properties

### 1.1 Overview

CRISPR-Cas9 gene editing has become one of the most disruptive technologies in scientific research. Its introduction has renewed hope for numerous genetic conditions once thought to be incurable. With a rapid transition toward translational applications, it has become evident that efficient delivery of the Cas9 cargo and significant gene editing efficiencies are crucial requirements for potential therapies.

Currently, many studies focus on either modifying Cas9 or developing new delivery systems. In contrast, the presently disclosed subject matter provides a more holistic approach to achieve these purposes. The presently disclosed methods aim to utilize the microenvironment, more specifically mechanical cues within the microenvironment, to improve delivery and editing efficiencies of CRISPR-Cas9.

By using substrates of varying stiffnesses, substantial increases in Cas9 gene editing were observed in comparison to standard tissue culture methods in a stiffness-dependent manner. The concept of using mechano-transduction to enhance gene editing was shown to be relevant across various hydrogel systems, transfection methods, forms of Cas9, gene targets and cell lines. Gene editing on hydrogels also resulted in a quicker gene expression and onset of gene editing. Perturbation of the cytoskeleton and nucleoskeleton reversed the effects of stiffness-mediated gene editing, thus establishing a critical link between mechano-transduction and gene editing.

Finally, mechanistic studies reveal that Cas9 reaches the nucleus quicker for cells cultured on hydrogels and that substrate stiffness increases chromatin accessibility to the Cas9 RNP. These findings provide unique and powerful solutions for the overall improvement of CRISPR-Cas9 gene editing and highlights the role mechanical cues may play in drug delivery and gene editing.

### 1.2 Background

The Cas9 cargo can be delivered as plasmid DNA, mRNA, or protein. Glass et al., 2018. Depending on its form, it must first be delivered to the cell, which is a challenge in itself. After this step, the mechanism will vary depending on the form as transcription and translation may be required to generate the Cas9 protein. Finally, all paths converge at the nucleus where the Cas9 and sgRNA ribonucleoprotein complex must travel and interrogate the chromatin prior to forming a double stranded break at the targeted sequence. Doudna and Charpentier, 2014; Sternberg et al., 2014. Numerous studies have aimed to improve the delivery of Cas9 through the design of novel vectors or modification of the Cas9 cargo itself in attempts to improve the gene editing process. Eoh and Gu, 2019.

Few researchers, however, have investigated how the cell microenvironment can be harnessed to improve Cas9 delivery and gene editing. Mechanical cues and more specifically, substrate stiffness could provide the means to do so. Though they may seem like disparate fields, mechanobiology and drug delivery often work in tandem with one another. Traditionally, it is known that therapeutic compounds, such as proteins and nucleic acids, must first enter the cell and undergo endosomal escape. From there, they will travel to the nucleus or to specific areas within the cell to perform their therapeutic purposes. The cytoskeleton, one of the driving forces of mechanobiology, is intricately involved throughout this entire process whether it be actin dynamics aiding in the nanoparticle uptake or microtubules and myosin facilitating transport. Ondrej et al., 2007; Coppola et al., 2013; Modaresi et al., 2018; Persson et al., 2013.

The cytoskeleton, along with other components also are responsible for converting mechanical stimuli into biochemical ones via mechano-transduction. For many mechano-transduction pathways, the forces are transmitted to the nucleus, which can lead to numerous outcomes, such as alterations to chromatin configuration or transient perturbations to the nuclear membrane. Cho et al., 2017. Further, several studies have shown that mechano-transduction can work synergistically with numerous biological processes, such as endocytosis, nuclear transport, transcription and cellular reprogramming. Modaresi et al., 2018; Huang et al., 2013; García-García et al., 2022; Tajik et al., 2016; Caiazzo et al., 2016.

### 1.3 Scope

This Example aims to establish the relation between mechanobiology and CRISPR-Cas9 gene editing. Specifically, this Example demonstrates that substrate rigidity provided by two-dimensional (2D) hydrogels can lead to quicker and more efficient gene editing. This principle holds across multiple hydrogel systems, transfection methods, forms of Cas9, target genes, and cell lines. It also is shown, through both small molecule inhibitor and siRNA studies, that the key mediators of mechano-transduction pathways, such as cytoskeletal and nucleoskeletal components, are directly involved in this phenomenon. Mechanistic studies reveal that Cas9 reaches the nucleus quicker for cells cultured on hydrogels and that substrate stiffness increases chromatin accessibility for the Cas9 RNP. These results suggest that the microenvironment could be a significant contributor to CRISPR-Cas9 gene editing and highlight its potential implications in gene therapy research.

### 1.4 Results

### 1.4.1 Matrix Stiffness Enhances CRISPR-Cas9 Gene Editing

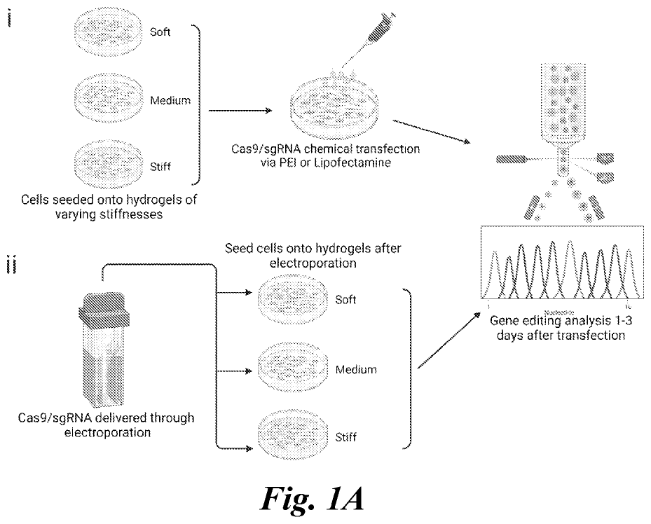

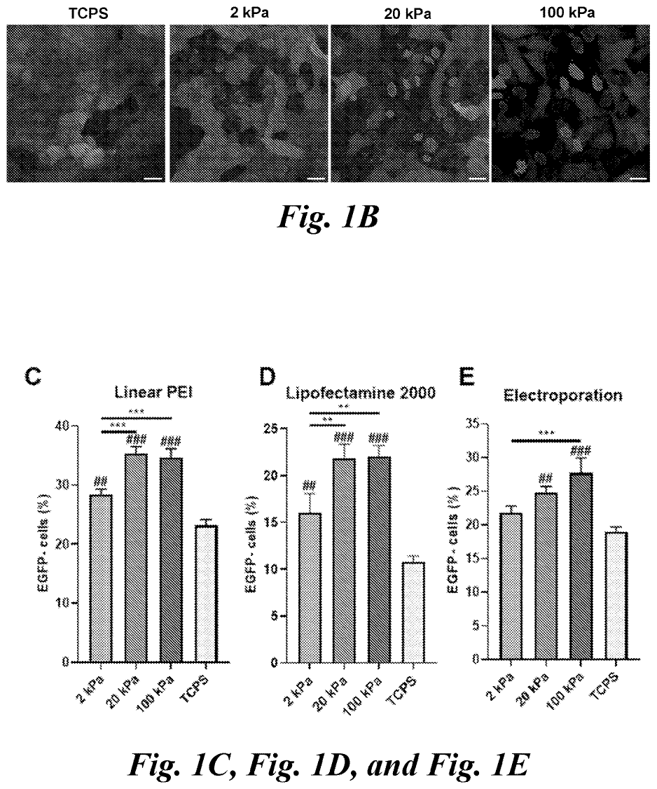

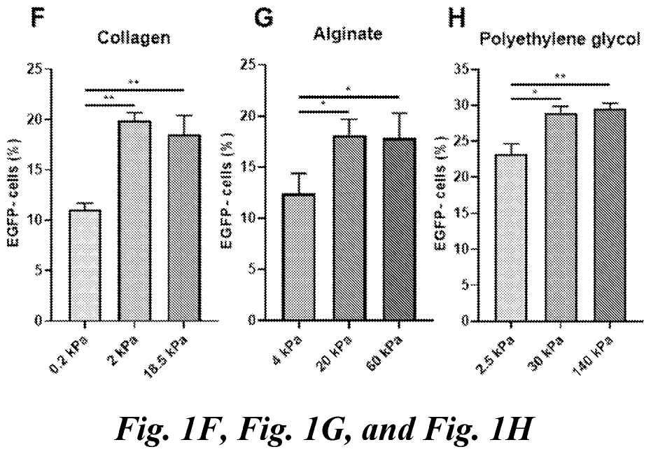

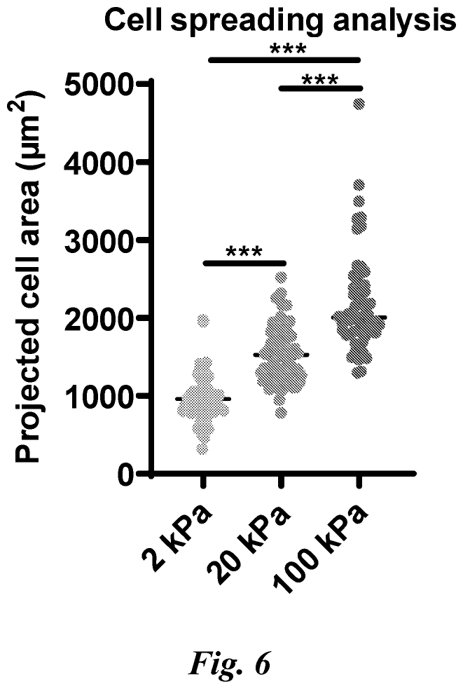

Previous studies have shown that increased cell contractility results in chromatin decondensation and increased force transmission to the nuclear lamina and chromatin. Jain et al., 2013; Le et al., 2016. Without wishing to be bound to any one particular theory, it was thought that increasing the stiffness of hydrogels could result in the same process, thus allowing for easier access of Cas9 to the chromatin and more efficient gene editing. In initial studies, U2OS.EGFP cells were cultured on polyacrylamide gels of varying moduli and transfected with the Cas9 and EGFP-targeting sgRNA plasmids complexed with linear polyethylenimine (PEI) (FIG. 1A). As a control, experiments also were performed in parallel with cells seeded on tissue culture polystyrene (TCPS) coverslips. Cell spreading was observed in a stiffness-dependent manner (FIG. 6) and images obtained through fluorescent microscopy demonstrate clear signs of gene editing indicated by the loss of fluorescence (FIG. 1B). Furthermore, a direct relationship between stiffness and gene editing was observed. Flow cytometry quantitatively validated these observations with all editing efficiencies for cells cultured on gels having significantly higher editing efficiencies than those cultured on TCPS with the highest editing efficiencies being close to 40% for the 20-kPa and 100-kPa conditions (FIG. 1C). Differences in editing also were significant when comparing the 2-kPa condition to the 20-kPa or 100-kPa conditions (FIG. 1C).

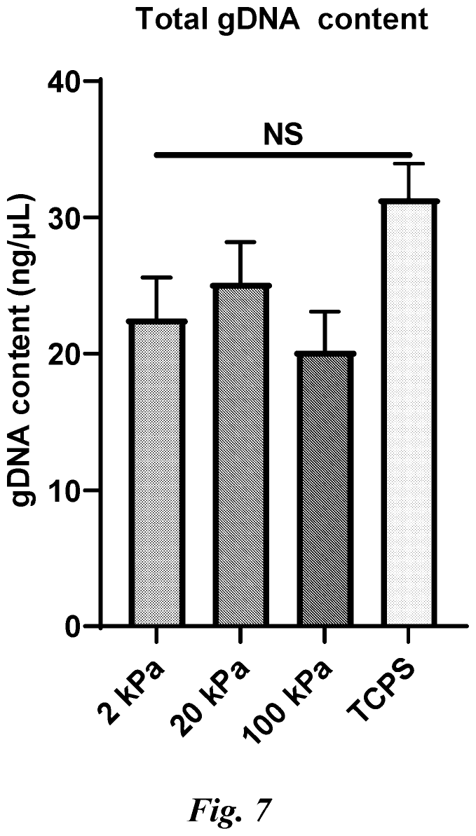

These results suggest that increasing the stiffness of the substrate enhanced gene editing. Because transfection efficiency could be related to the proliferation rate, genomic DNA (gDNA) was quantified at the conclusion experiment. Measurements of gDNA content verified that there were no significant differences amongst the conditions, thereby further strengthening the assertion that the differences are from the stiffness (FIG. 7). To ensure findings were not exclusive to PEI transfections, experiments were repeated with other commonly used transfection methods, such as Lipofectamine™ 2000, i.e., a 3:1 mixture of DOSPA (2,3-dioleoyloxy-N-[2 (sperminecarboxamido)ethyl]-N,N-dimethyl-1-propaniminium trifluoroacetate) and dioleoylphosphatidylethanolamine (DOPE)) and electroporation. When using Lipofectamine™ 2000, the same trends were maintained (FIG. 1D), though the editing values were lower than those obtained with PEI. For electroporation, however, we did not observe a significantly higher editing efficiency for the 2-kPa condition compared to the TCPS sample and only the 100-kPa condition had significantly higher gene editing than the 2 kPa condition (FIG. 1E). This observation could be explained by further delving into the experimental parameters. For studies using Lipofectamine™ 2000 and PEI, cells are first allowed to adhere prior to transfection. By the time of transfection, cells on the gels will have formed focal adhesions due to increased interactions with the covalently bound adhesive ligands found on the gels. Ventre and Netti, 2016.

These established focal adhesion complexes, as evidenced later, play a crucial role in the gene editing process. For the electroporation process, it may be more difficult to observe the contributions of the substrate as the Cas9 cargo is already within the cell when it is attempting to form mature focal adhesion complexes (FIG. 1A). Despite this difficulty, electroporation will provide several advantages and insightful observations for this study when developing a potential mechanism. Editing efficiencies in intermediate and stiff hydrogels were still higher with electroporation than those of the TCPS condition. This observation could suggest that, for these conditions, nanocomplexes are able to more efficiently use the actin network and microtubules to transport to the nucleus, thereby leading to quicker editing. Rosazza et al., 2016. In addition to using different transfection methods, three commonly used hydrogel systems also were employed to rule out the possibility that these findings were not limited to just any one biomaterial system. Li et al., 2017.

For collagen, alginate, and polyethylene glycol (PEG), all systems demonstrated a significant gain in editing efficiency with increased stiffness (FIG. 1 F-FIG. 1H). Across four materials systems with vastly different chemistries, mechanical properties, and cell binding ligands, it is evident that stiffness can enhance gene editing. For all subsequent experiments, the PAM hydrogel system and PEI transfection method were used as this combination gave the highest editing efficiencies while maintaining the trend amongst the stiffnesses. Overall, the preceding data confirm that gene editing significantly improves with substrate stiffness.

1.4.2 Stiffness from Hydrogels Confer Rapid Cas9 Expression and Gene Editing

More efficient nucleocytoplasmic shuttling, leading to quicker and higher gene editing, is a possible reason for the increased editing efficiencies on hydrogels. To further investigate this hypothesis, the kinetics of the Cas9 gene expression and gene editing process were investigated. Transfections were performed with mCherry-Cas9 and sgRNA plasmids to investigate if differences in Cas9 expression could be observed between the hydrogels and TCPS condition and among the hydrogel groups.

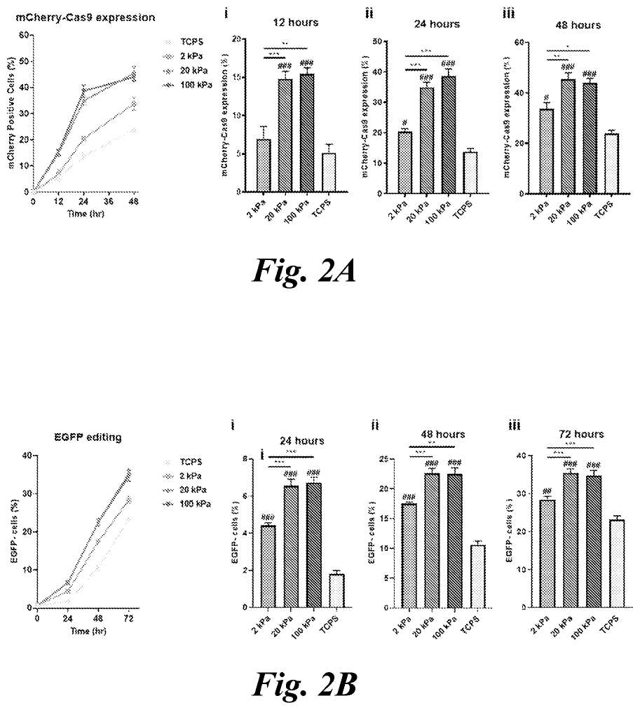

For all conditions, the percentage of mCherry-expressing cells increased, indicating sustained Cas9-mCherry expression throughout the experiment (FIG. 2A). As early as 12 hours, there was significantly more mCherry-positive cells in the 20-kPa and 100-kPa samples compared to the TCPS and even 2-kPa hydrogel conditions (FIG. 2A(i)). After 24 hours, mCherry-Cas9 expression increased in all conditions, all hydrogel groups had significantly higher mCherry-Cas9 expression than TCPS control, and the trend in stiffness remained consistent (FIG. 2A(ii)). By 48 hours, mCherry-Cas9 expression further increased and all hydrogel groups again demonstrated a higher mCherry-Cas9 expression compared to the TCPS group (FIG. 2A(iii)). For the 20-kPa and 100-kPa conditions, mCherry-Cas9 expression approached 50%.

The editing efficiency also was quantitively assessed via flow cytometry at various timepoints to explore if the increase in mCherry expression correlated to an increase in editing efficiency. To account for the latency occurring between Cas9 expression and actual editing, the timepoints of 24, 48, and 72 hours were chosen. Throughout the experiment, the overall editing efficiency increased with time in a manner similar to the mCherry-Cas9 expression (FIG. 2B). Beginning at 24 hours, editing efficiencies for all conditions were expectedly low. Despite this observation, all hydrogel samples had significantly higher percentages of EGFP-negative cells and this value increased with the stiffness (FIG. 2B(i)). These trends remained consistent at the 48-hour and 72-hour timepoints with the only difference coming from the increases in editing efficiencies found in all groups (FIG. 2B(ii) and 2B(iii)). From the Cas9-mCherry studies, it is evident that substrate stiffness provided by the hydrogels resulted in quicker expression of Cas9-mCherry, which is consistent with previous literature demonstrating that transcription can occur rapidly upon direct stretching of the chromatin. Tajik et al., 2016.

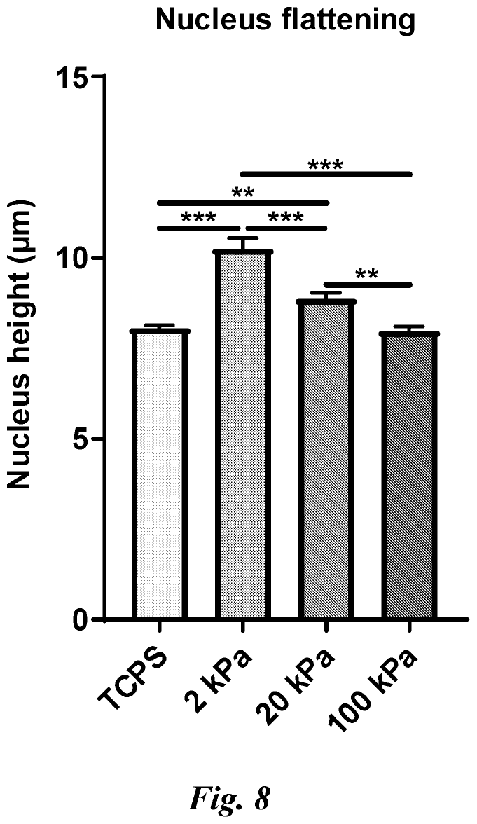

For our purposes, the PAM 2D system also is capable of imparting passive stretch to the nucleus, thus potentially allowing for quicker transcription of the Cas9-mCherry plasmid (FIG. 8). The rapid and increased expression in Cas9 was further supported by the increases in editing efficiency with time. The trends in Cas9 expression and editing efficiency were consistent with one another and appear to further support the notion that the quicker expression of Cas9 caused by the hydrogel stiffness could result in increased gene editing. Between the two studies, it is important to note the slight decrease going from the mCherry-Cas9 expression to the actual gene editing percentage. This decrease could be due to the biological processes required to go from one to the other, such as interaction with the sgRNA to form the RNP complex, transport back into the nucleus, and finally probing the target sequence. From these studies, it is apparent that the stiffness provided by the hydrogel substrates allows for quicker Cas9 expression and editing.

1.4.3 Stiffness-Enhanced Gene Editing Also is Observed with Other Forms of Cas9, Gene Targets, and Cell Types

The plasmid form of Cas9 must first undergo transcription and translation prior to traversing the nucleus. Despite this limitation, we were still able to demonstrate the enhanced effects of substrate stiffness on gene editing. To show that these effects were not specific to any of the biological processes involving the Cas9-plasmid, we examined editing efficiencies using Cas9 in the mRNA and protein forms. For transfections using either the Cas9 mRNA or Cas9 RNP, quicker editing was observed and editing efficiencies were higher than those obtained using plasmid Cas9 as expected. Lin et al., 2022.

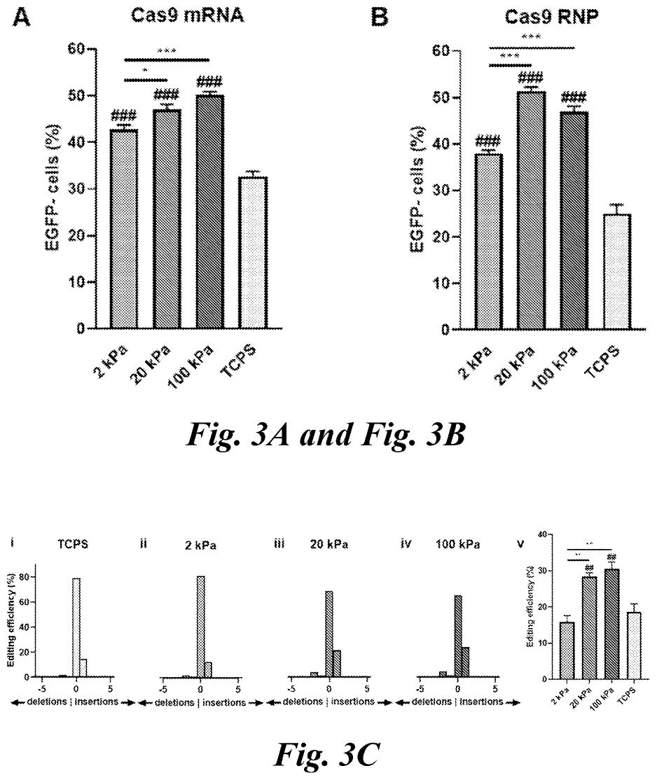

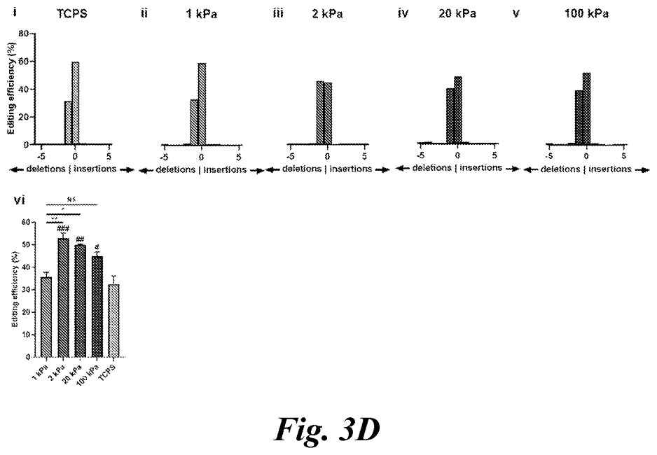

For Cas9 mRNA, editing efficiencies on the hydrogel samples were significantly higher than those obtained on TCPS (FIG. 3A). Within the hydrogel conditions, editing efficiencies were significantly higher in cells cultured on 20-kPa and 100-kPa hydrogels when compared to those in 2-kPa hydrogels, much like the plasmid-Cas9 experiments. For Cas9 RNP studies, the editing efficiencies and trends were like those found in the Cas9 mRNA studies with the hydrogels outperforming the TCPS control and the editing efficiency increasing with stiffness (FIG. 3B). With these findings, it appears that substrate stiffness enhances the gene editing for all forms of Cas9. Though the editing values were higher for the Cas9 mRNA and RNP studies, the trends were identical to those observed in the plasmid-Cas9 studies. By using alternative forms of Cas9, we are able to expand the relevancy of mechano-transduction to CRISPR-Cas9 gene editing as many studies prefer the Cas9 mRNA/protein for in vivo or ex vivo applications due to the reduced risk of off-target editing and quicker onset of gene editing. Lin et al., 2022; Xu et al., 2021; Behr et al., 2021; Tong et al., 2019. Additional clues also are presented as to the role mechanobiology may play in the gene editing process whether it be in aiding in the translation of the Cas9 mRNA or the nuclear transport of the Cas9 RNP.

To demonstrate the potential for therapeutic gene editing, the AAVS1 locus was selected as an endogenous gene target. For these studies, the editing efficiencies were calculated via TIDE analysis of the Sanger sequencing data. Brinkman et al., 2014. Overall, the gene editing data of the AAVS1 gene in the U2OS.EGFP cell line closely matched the EGFP editing data obtained from flow cytometry (FIG. 3C (v)). Editing efficiencies on the 20-kPa and 100-kPa gels were approximately 30% and were significantly higher than editing on both the TCPS and 2-kPa conditions. One difference of note in the analysis of Sanger sequencing traces was that the editing efficiency in the 2-kPa condition was not higher than the TCPS. This observation could be due to several reasons including the variation between gene targets, its location within the chromatin or perhaps even the method for analyzing the editing. Sansbury et al., 2019; Javaid and Choi, 2021.

When analyzing the types and frequency of edits made in the U2OS cell line, it appears that insertions seems to be the predominant mechanism of DNA repair and this frequency actually increases with stiffness (FIG. 3C (i-iv)). This observation is interesting as it has been shown that the predominant form of repair in the non-homologous end joining pathway (NHEJ) is deletions and ligation of the resultant strands. Wang et al., 2014. The ability to increase the frequency and likelihood of +1 bp insertion via substrate stiffness is of great significance as it could reduce the likelihood of harmful frameshift mutations, further promote accurate NHEJ, and allow for proper DNA repair without the need for a donor template. Guo et al., 2018; Bermudez-Cabrera et al., 2021.

Human mesenchymal stem cells (MSCs) were chosen as an additional cell line to demonstrate the gene editing capabilities of this system on a known hard-to-transfect cell line. de Carvalho et al., 2018; Hamann et al., 2019. MSCs also have shown great potential for cell therapies due to their vast differentiation potential and multitude of roles in vivo. Manotham and Chattong, 2018; Golchin et al., 2020. For the gene editing of MSCs, the Cas9 mRNA was used due to its previous success in primary cell lines and reduced cytotoxicity. Hendel et al., 2015.

Furthermore, as mechano-sensing varies across different cell lines, the stiffness range tested was adjusted for the gene editing of MSCs (FIG. 3D). Janmey et al., 2020. Even with a different cell line and stiffness range, the trends observed were similar to those in the U20S cell line. Editing efficiencies on the 2-kPa, 20-kPa, and 100-kPa gel conditions were again significantly higher than those found in the TCPS and 1-kPa conditions (FIG. 3D(vi)). For the gene editing of MSCs, however, the 2 kPa condition was found to be the optimum stiffness. When analyzing the frequency and mechanism of DNA repair, deletions were found to be the predominant mechanism (FIG. 3D(i-v)). Numerous factors, such as the cell type, and chromatin environment, could account for this observation. Brinkman et al., 2014. Though stiffness in itself could not improve the frequency of +1 bp insertions despite slightly higher frequencies in the hydrogel conditions, it could be used in conjunction with other methods proven to promote the insertion mechanism of NHEJ. Bermudez-Cabrera et al., 2021.

Overall, the ability to demonstrate similar effects with multiple forms of Cas9, cell types and gene targets allows us to further demonstrate the effectiveness of mechanobiology in aiding CRISPR-Cas9 gene editing, as well as its relevancy to numerous other biological processes including translation and nuclear shuttling.

### 1.4.4 Perturbation of the Cytoskeleton, Nucleoskeleton and Mechano-Transduction Pathways Impacts Gene Editing

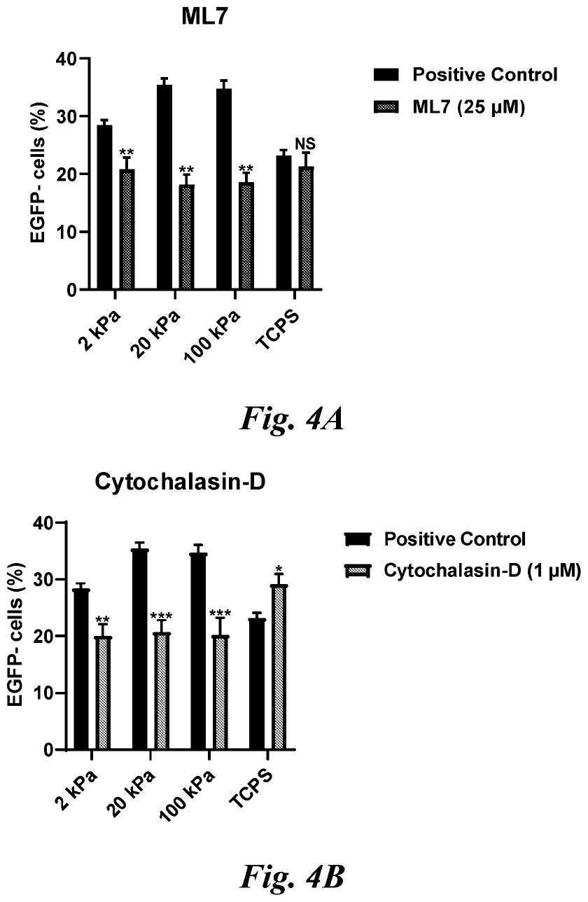

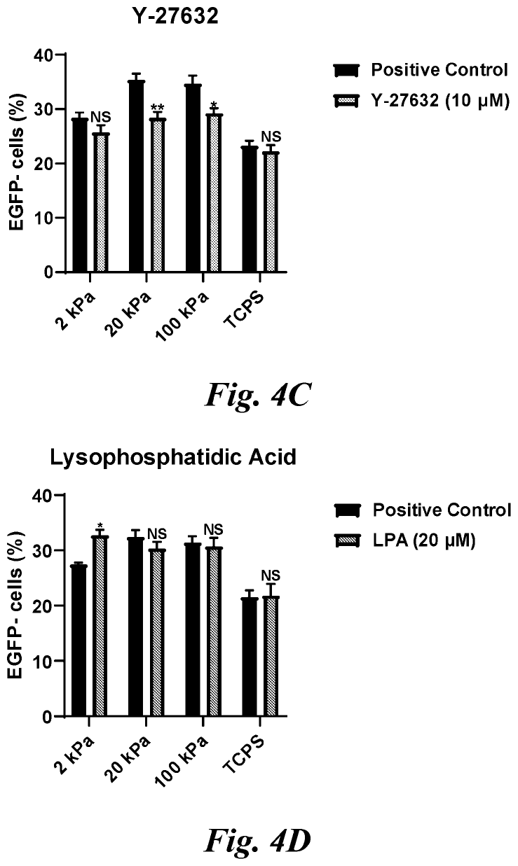

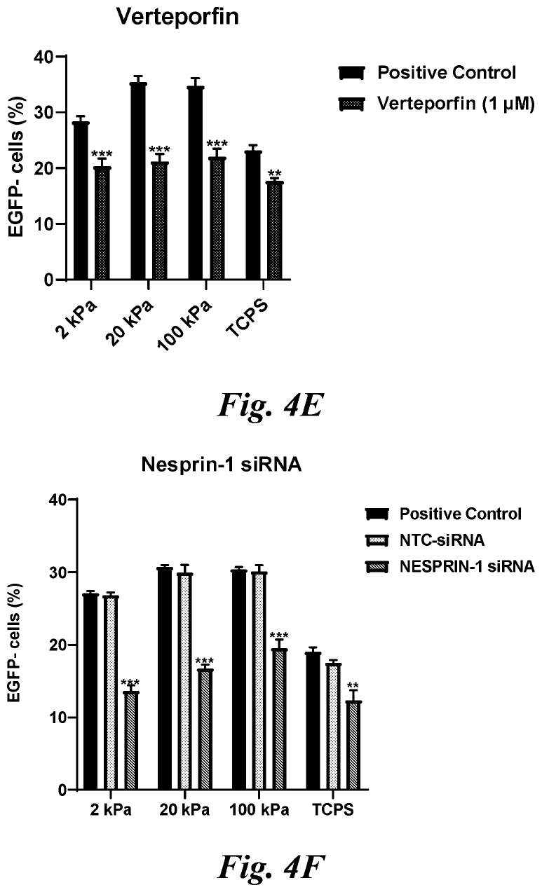

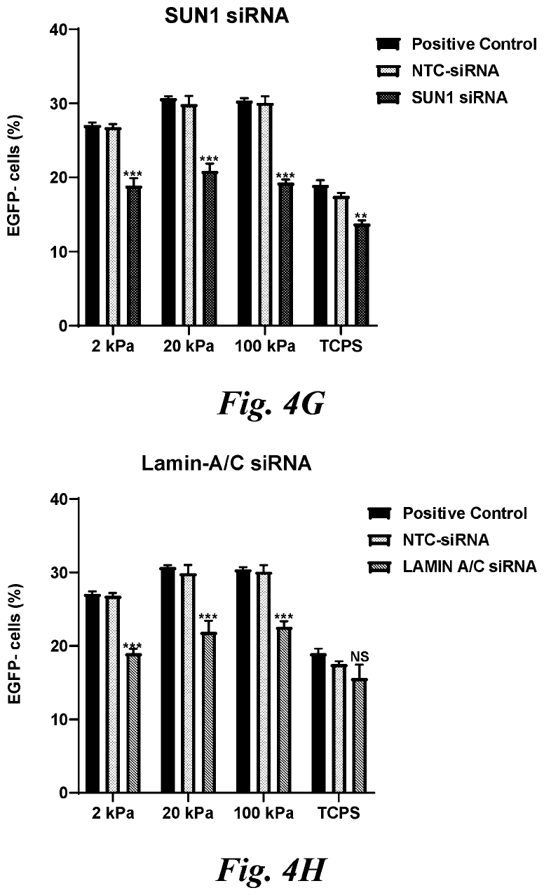

In an effort to gain a general understanding of how substrate stiffness enhances gene editing, various small molecules and biologics were used to elucidate the role of key mechano-transduction constituents in gene editing. The first inhibitor tested was ML7, an inhibitor of myosin light chain kinase (MLCK). Shi et al., 2007. In all hydrogel samples, the treated samples had significantly reduced editing efficiencies in comparison to the non-treated controls (FIG. 4A). For the TCPS condition, however, no difference was observed between treated and untreated. Furthermore, the increase in editing observed with stiffness also was lost upon inhibition of MLCK with all treated samples showing no significant differences amongst one another. As MLCK is a key regulatory element of type II myosins, its inhibition results in reduced actin-myosin interactions and thus a reduction in cytoskeletal tension. Cai et al., 1998; Chen et al., 2014. The decreased cytoskeletal tension could have multiple outcomes, such as inefficient nucleocytoplasmic shuttling, lower transcription rate, and reduced chromatin accessibility. Tajik et al., 2016; Neco et al., 2004; Cho et al., 2017. All of these situations could reverse the effects of substrate-mediated gene editing.

The effects of actin were investigated by treating cells with cytochalasin-D to inhibit actin polymerization. Though the trends were similar to those found with ML 7 treatment, an interesting observation was a significant increase in editing efficiency in the TCPS condition after treatment with cytochalasin-D (FIG. 4B). Like the ML7-treated samples, the trend in relation to stiffness is reversed and editing efficiencies closely resemble that of the TCPS positive control sample. These results further indicate that actomyosin interactions are a crucial component of the substrate-mediated effects on gene editing. For the TCPS treated condition, several explanations exist for the increase in editing efficiency. The diffusion of intracellular cargo, such as nanocomplexes, may be difficult due to the actin network occupying such a large volume within the cell. Grady et al., 2017. Therefore, increasing the free volume of the cell by inhibiting F-actin polymerization could possibly increase the diffusion of nanoparticles to the nucleus and as a result, the editing efficiency.

Inhibition of F-actin polymerization, however, was found to have a deleterious effect on gene editing for hydrogel samples. The differences in effects could be due to the fact that it is challenging to make direct comparisons between hydrogel culture and conventional tissue culture as the two are vastly different from one another. When purely looking at the stiffness, TCPS is a stiffer substrate than the hydrogel samples. Despite this observation, numerous other factors come into play with 2D hydrogel culture, such as the type and spatial distribution of binding ligands. These effects could play additional roles to enhance gene editing and could be reversed when treated with cytochalasin-D.

An alternative explanation that could apply for both TCPS and hydrogels comes from the cytoskeletal arrangement as transport via diffusion alone is only applicable to certain small molecules, such as ATP. Snider et al., 2004. It is known that culturing cells on hydrogels allow for increased alignment of cytoskeletal components due to the even distribution of binding ligands. Gupta et al., 2015; Kechagia et al., 2019. This feature is not found in conventional tissue culture and thus results in cells with randomly orientated cytoskeletal structures. Caliari and Burdick, 2016. It also is known that the transport of intracellular cargo requires the cytoskeletal network. Ondrej et al., 2007; Snider et al., 2004; Khaitlina, 2014; Lieleg et al., 2010. The increased cytoskeletal alignment found in the hydrogel samples could lead to a more direct route to the nucleus via the microtubule and/or the actin network leading to quicker and higher overall gene editing than editing on TCPS.

The contributions of the Rho pathway were investigated using the small molecule inhibitor Y-27632. Ishizaki et al., 2000. Significant differences between the treated and untreated groups could only be seen at the 20-kPa and 100-kPa conditions and the trends seen with stiffness also were reversed with treatment (FIG. 4C). These results seem to be consistent with other findings as Rho activity is reduced on softer substrates. Hoon et al., 2016.

One of the major downstream processes controlled by the ROCK1 is actomyosin contractility. It would seem that inhibition of this process should lead to similar results as those obtained with ML7 and cytochalasin-D treatment but analyzing the mechanism of action of these inhibitors could address this issue. Both ML7 and cytochalasin-D directly act upon their respective constituents thus leading to a direct response. Y-27632 acts upon ROCK1 and even though this pathway is responsible for actomyosin contractility, its effects are further downstream. As a result, the effects of ROCK1 inhibition may not be as discernable due to an attenuated response. This hypothesis is further supported by the data as the overall editing values for the treated samples are higher than those obtained with treatment with cytochalasin-D or ML7. Based on these observations, it seems that ROCK1 is more involved for the intermediate and stiffer hydrogels as a result of increased Rho activity on stiffer substrates.

Lysophosphatidic acid (LPA) is a potent activator of RhoA, located upstream of ROCK1. Amerongen et al., 2000. As the effects of Y-27632 treatment were only noticeable on stiffer substrates, LPA was used in an effort to see if activation of the Rho pathway could possibly increase the editing efficiency on softer substrates and perhaps even the efficiencies on stiffer substrates. Doing so also highlights the potential use of small molecules to stimulate mechano-transduction pathways to enhance gene cleavage. It was found that only the 2-kPa condition has a significant increase in editing (FIG. 4D). For the other conditions, little to no difference was observed between the treated and untreated groups. As provided hereinabove, Rho activity is reduced on softer substrates. Thus, it would be plausible that increasing Rho activity on softer substrates would make the editing values comparable to that of the stiffer conditions. These results seem to agree with this assertion. These results also suggest that the ROCK1 pathway only impacts the gene editing to a certain extent more specifically on softer substrates. From both Y-27632 and LPA treatment studies, it is apparent that other cytoskeletal components play a bigger role the stiffness-mediated enhancement of gene editing. Alteration of the RhoA/ROCK1 pathway could be too broad to detect within the context of gene editing as many downstream pathways are related to RhoA and ROCK1, many of which overlap.

As many mechanical cues detected by the cell converge to the YAP/TAZ pathways, their contributions to the hydrogel-mediated gene editing process also were investigated. Panciera et al., 2017. The small molecule drug, Verteporfin, was used due to its widely reported use as a YAP inhibitor. Wang et al., 2016. After treatment, a significant decrease in gene editing was found in all samples, including the TCPS condition (FIG. 4E). In addition, there were no significant differences amongst the conditions. These observations seem to suggest that the YAP/TAZ pathways are involved in the editing process for all samples. This observation is of note as it is generally understood that under certain conditions, such as a soft substrate, YAP/TAZ signaling cannot occur due to its sequestration in the cytoplasm. Panciera et al., 2017; Kurotsu et al., 2020.

As stiffness increases, YAP and TAZ can enter the nucleus and activate subsequent pathways. By this understanding, there should be sufficient YAP/TAZ activation in the 20-kPa, 100-kPa and TCPS conditions due to stiffness. Therefore, it would be feasible that the inhibition of YAP would result in a decrease in editing for these conditions. One would not expect to see a reduction in editing efficiency for the 2-kPa condition due to it being relatively softer. Despite this result, a significant difference was still observed. There could be several explanations to this finding including the context-dependent meaning of a soft substrate. As many different cell types detect different ranges of stiffnesses it could be that 2-kPa is sufficient for YAP nuclear shuttling in the U2OS cell line. Another explanation could be the presence of a broad non-specific effect. As YAP/TAZ are implicated in numerous biological processes the inhibition of these pathways could lead to many downstream effects all of which could result in reduced gene cleavage. Many types of mechanical cues are converted into biochemical signals in the form of YAP/TAZ within the cell. YAP/TAZ plays a role in gene editing though its exact contributions are unknown.

Mechano-transduction converts mechanical stimuli from the extracellular domain into biochemical ones within the cell which are then transmitted to the nucleus. How these signals are transmitted from the cytoplasm to nucleus is of great interest. The linker of nucleoskeleton and cytoskeleton (LINC) complex plays a crucial role in this process. The LINC complex consists of the Nesprins, and SUN dimer which interacts with the nuclear lamins. Uhler and Shivashankar, 2017.

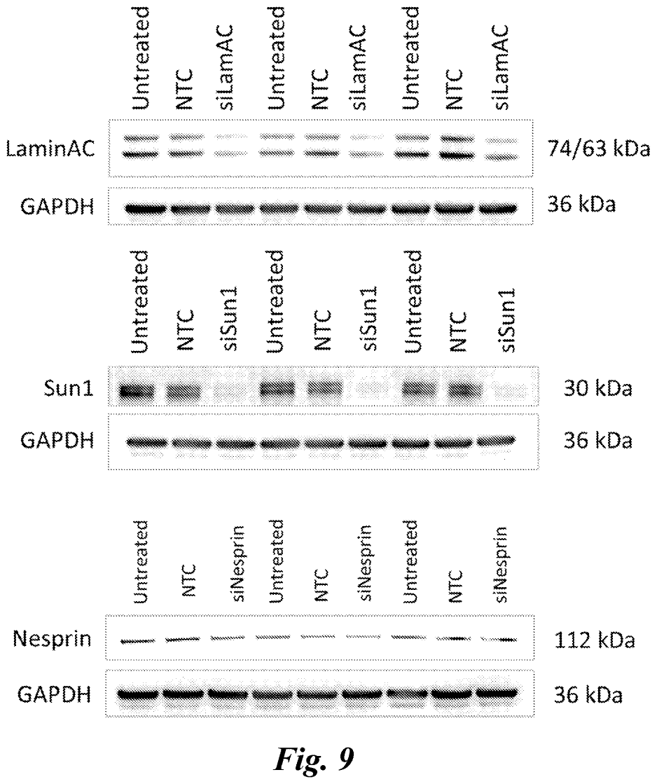

Further investigation of how the LINC complex contributes to gene editing could provide crucial insight into the mechanism of gene editing, as well as the nucleus and accompanying nucleoskeleton. To achieve specific targeting of each component of the LINC complex, siRNAs were chosen to perform knockdown studies (FIG. 9). Inhibition of both components of the LINC complex led to a significant decrease in editing compared to the NTC-siRNA treated conditions, but several key differences were observed between Nesprin-1 and Sun1 knockdown studies. Nesprin-1 is present in the cytoplasmic compartment and interacts with microtubules, intermediate filaments or actin. Uhler and Shivashankar, 2017; Zhang et al., 2007.

Nesprin-1 knockdown led to significant decreases in editing efficiency for all samples (FIG. 4F). Some trends remained, however. Specifically, the trend in stiffness was maintained amongst the hydrogels and the 20-kPa and 100-kPa conditions still had significantly higher editing efficiencies than that found in TCPS. These observations seem to suggest that the knockdown of Nesprin-1 leads to a non-specific decrease in gene editing. This finding could be due to Nesprin-1 interacting with multiple cytoskeletal components or the presence of other Nesprins, all of which could contribute to gene editing. Because of this observation, it seems that Nesprin-1 plays a role in gene editing but perhaps in a broader context applicable to both TCPS and hydrogel culture. When knocking down SUN1, significant decreases in editing were again found in all conditions, but no significant differences found amongst the hydrogel samples (FIG. 4G). All hydrogel groups also had significantly higher editing than TCPS after siRNA treatment. The SUN dimer rests in the inner nuclear membrane and bridges the cytoskeleton and nucleoskeleton. It senses the overall stiffness felt in the cytoplasm and transmits it to the nucleus. Using siRNA to knockdown SUNI would impair the nuclei's ability to sense the stiffness in the cytoplasm, thus reversing the trend amongst the hydrogel conditions as reported. When comparing the editing efficiencies of TCPS and hydrogels, it is evident that other factors could be at play aside from the stiffness. Nuclear lamins, such as Lamin A/C, are intermediate filaments that comprise a large portion nucleoskeleton and are responsible for regulating the physical properties of the nucleus. Lammerding et al., 2006.

Treatment with LMNAC-siRNA resulted in significant reductions in editing efficiency for all hydrogel samples, but not for the TCPS condition (FIG. 4H). One of the drawbacks of culturing cells on TCPS is the irregular and heterogeneous distribution of cell binding ligands that come from interactions between TCPS and proteins found in serum or produced by the cells. Caliari and Burdick, 2016; Lerman et al., 2018. This drawback leads to improper focal adhesion development and spacing, causing reduced force transmission to the nucleus thus reducing the involvement of the nucleoskeleton. Kechagia et al., 2019. Such a reason could be why editing efficiencies on gels were impacted while not on TCPS. Even with this reduction, editing efficiencies in all hydrogel groups were higher than that found on TCPS. Within the hydrogel groups, a significant difference could still be found between 2 and 100 kPa (p<0.05). These findings are more than feasible if we look at the overall editing process presented thus far.

We have shown that the editing process on hydrogels is a coordinated effort of the cytoskeleton, mechano-transduction pathways, and the nucleoskeleton. It is more than likely that there also are many other components involved not discussed in this Example. The nucleoskeleton itself is a complex environment with many other constituents including spectrins, titins, nuclear actin, myosins, and even B-type lamins not targeted during the siRNA treatment. Simon and Wilson, 2011; Burke and Stewart, 2013. All of these components have been shown to interact with the LINC complex or with the chromatin itself. It is possible that the targeted knockdown of LMNAC could lead to the involvement of these other components, thus still preserving some of the features of editing on hydrogels, such as differences in editing between some hydrogel stiffnesses and the overall higher editing seen on hydrogels versus TCPS. Even with the possible involvement of these other elements, however, the noticeable decrease in editing efficiency after LMNAC knockdown suggests that it still plays a critical role in substrate-mediated gene editing.

### 1.4.5 A Feasible Mechanism for the Substrate-Mediated Improvements in Gene Editing

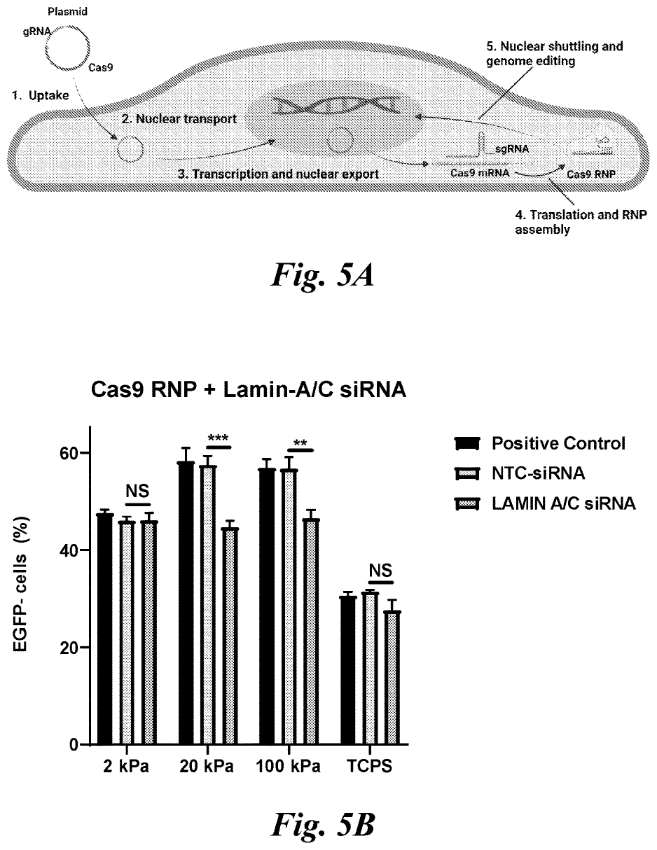

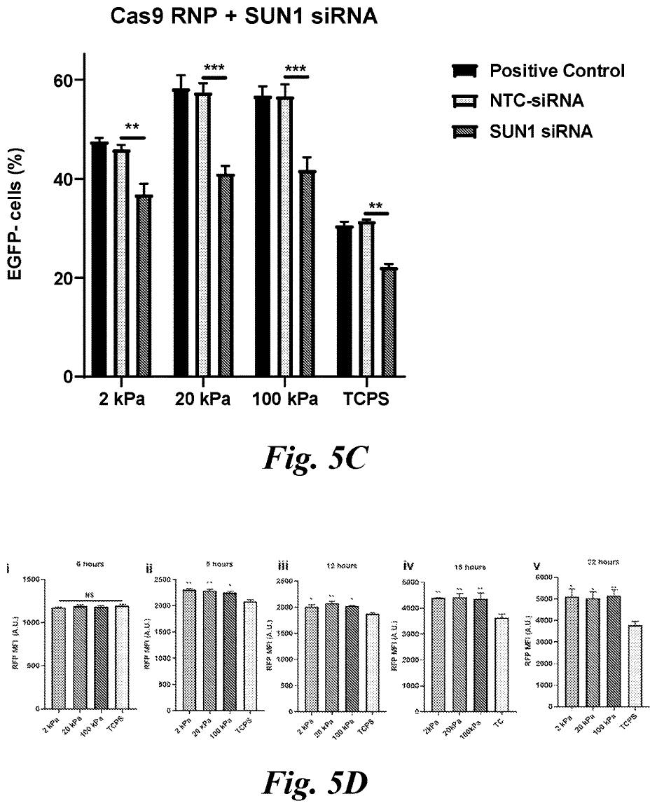

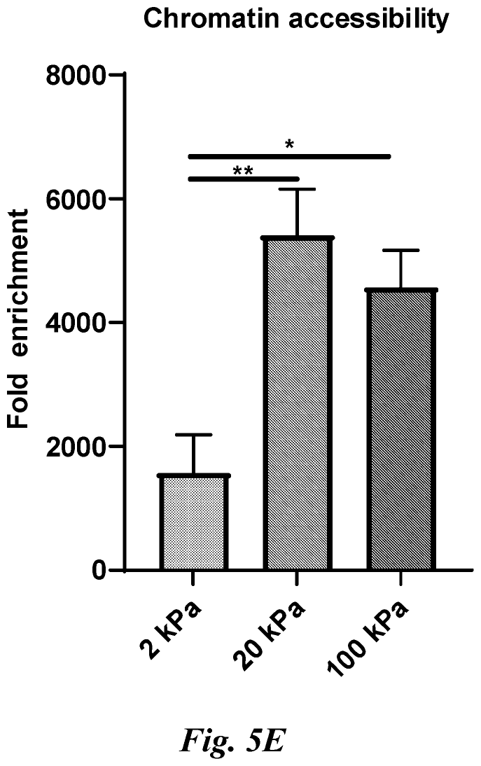

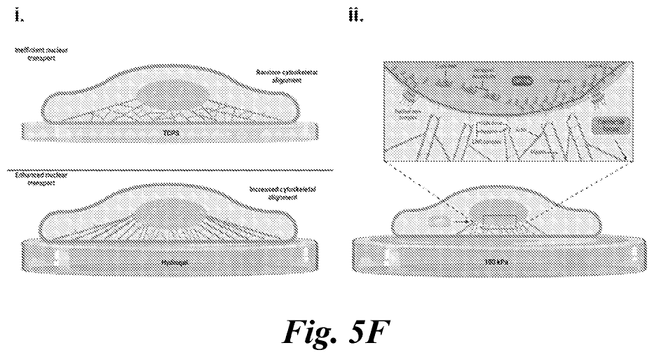

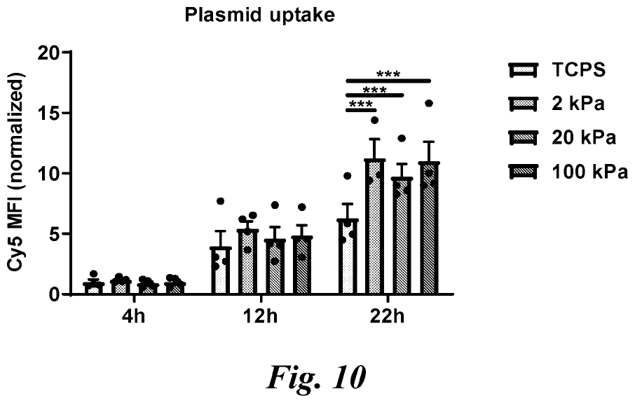

The results from the inhibitor studies provided critical information for elucidating the mechanism behind these findings. The steps of the CRISPR delivery process for the plasmid form are quite involved and include uptake, transport into the nucleus, transcription/nuclear export, translation and RNP formation, and finally, transport back into the nucleus for genome editing (FIG. 5A). Although it is possible all areas could be improved by the substrate stiffness, we focused on uptake and nuclear transport as these steps are the first and last steps of the plasmid's involvement in the process. For these studies, the Cas9 and sgRNA plasmids were labeled with Cy5 dye. When assessing the uptake via flow cytometry, significant differences amongst the various conditions could not be observed until 22 hours (FIG. 10). At this stage, all hydrogel groups exhibited higher mean fluorescence intensities (MFI) than the TCPS group, indicating higher intracellular plasmid concentrations. Despite this finding, we could not observe significant changes within the hydrogel groups. These findings are in contrast to what has been reported although these studies were performed using a different cell type, transfection platform, cell culture substrate, and plasmid. Modaresi et al., 2018.

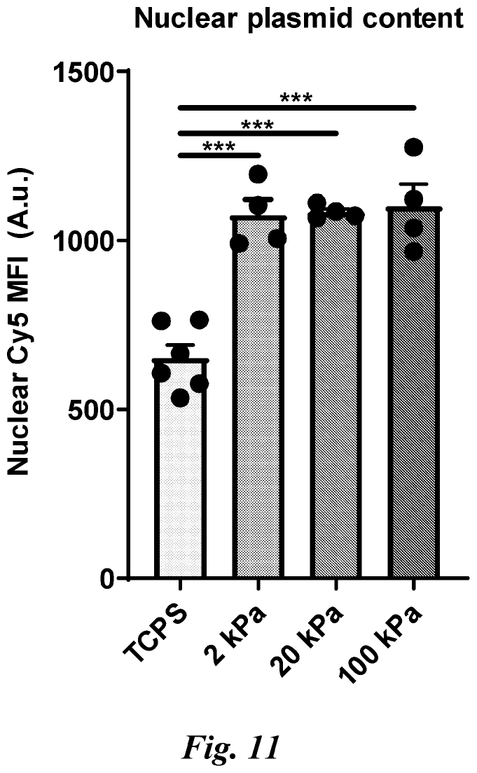

From these data, it seems that the uptake is affected by the hydrogels, but not at a stage in which stiffness may play a direct role. For nuclear transport, cell nuclei were isolated and analyzed via flow cytometry for Cy5 signal. A similar trend was observed with the hydrogels exhibiting higher Cy5 intensities within the nuclei, but no significant changes among hydrogel conditions (FIG. 11). At these two stages, it is evident that the hydrogels have an effect, but the effects of stiffness are still not evident.

Several possibilities exist for this effect and one involves the surface chemistry of the substrates. The two main features that distinguish the hydrogels from TCPS is the substrate stiffness, as well as the even distribution of ligands presented by the ECM coating. Both are necessary for proper focal adhesion development and maintenance. It is possible that at these stages, the surface chemistry plays a larger role than stiffness, thus explaining the higher values on hydrogels versus TCPS, but a lack of discernible trend between the hydrogel conditions.

siRNA treatments for the LINC complex also were performed with Cas9 RNP transfections to see alterations to the nucleoskeleton would directly impact the Cas9 protein's nucleocytoplasmic shuttling and editing capabilities. Lamin A/C and SUN1 knock down of these targets prior to Cas9 plasmid transfection resulted in most if not complete reversal of the trend seen with stiffness. Cas9 RNP transfection following Lamin A/C siRNA treatment led to a significant decrease editing in only the 20-kPa and 100-kPa conditions (FIG. 5B). No significant differences were observed, however, in editing amongst the hydrogel stiffness after siRNA treatment. Recall in the siRNA/plasmid Cas9 studies, a significant difference was still observed between the 2-kPa and 100-kPa conditions. The loss of trend observed in the Cas9 RNP transfections show that stiffness is involved for Cas RNP transport to the nucleus, and its interactions with Lamin A/C are a key driving force. It must be noted, however, that the reductions in editing were not as dramatic as those seen with the plasmid Cas9 studies. There is a possibility that Lamin A/C plays a larger role for the plasmid form of Cas9. Similar to the results shown from the siRNA/plasmid Cas9 transfections, Cas9 RNP transfection following SUN1 knockdown resulted in a significant difference in editing efficiency for all groups though to a lesser extent (FIG. 5C). This result further demonstrates that SUNI plays a critical role in the nuclear transport of Cas9 regardless of the form of Cas9, as well as the culture condition. This finding is consistent with previous reports demonstrating SUNI's involvement in transport of cargo through the nucleus. Li and Noegel, 2015; Liu et al., 2007.

The next experiments focused on nuclear transport of the Cas9 RNP to see stiffness accelerated this process, resulting in higher gene editing. For these experiments, cells were electroporated with Cas9-RFP and sgRNA. The use of electroporation allows us to ignore the contributions of the uptake phase and focus our attention on the nucleocytoplasmic shuttling and editing processes. Rosazza et al., 2016. From here we can better elucidate the stages at which substrate stiffness enhances gene editing. At various timepoints, the cell nuclei were isolated, and flow cytometry was performed to quantify the RFP MFI as an indicator of Cas9 present in the nucleus. At 6 hours, no changes were observed (FIG. 5D(i)), but a trend became apparent soon after at 9 hours (FIG. 5D(ii)) with the hydrogel samples exhibiting higher RFP MFI values than the TCPS condition. Additional measurements were taken at 12 and 15 hours to see if a trend would be observed among the different stiffnesses, but none were observed and the hydrogel conditions continued to maintain higher RFP signal in the nuclei compared to TCPS (FIG. 5D(iii) and FIG. 5D(iv)). This trend further held at 22 hours where the difference in nuclear RFP signal between hydrogels and TCPS increased, indicating efficient nuclear transport on hydrogels leads to increased accumulation of Cas9 and a higher potential for gene editing (FIG. 5D(v)).

As provided hereinabove there could be several reasons as to why there is a difference between TCPS and the hydrogels, but no trend with stiffness with surface chemistry being presented as a possibility. An alternative explanation relates to the nucleus. The nuclear pore complex (NPC) is the gatekeeper of the nucleus, responsible for the import of various macromolecules including ribonucleoproteins to the nucleus. Bai et al., 2017. For a plasmid or protein to enter the nucleus, it must first traverse various cytoskeletal components including actin and the microtubules to reach the nucleus where it uses its own nuclear localization signals (NLS) or “piggybacks” off various nuclear proteins or transcription factors to enter the nucleus through the NPC in a NLS- and importin-dependent fashion. Bai et al., 2017. Increased involvement of cytoskeletal components conferred by the hydrogels may aid in the travel to the nucleus, but the nuclear traversal may be a bottleneck for both the Cas9 plasmid and RNP. It is known that stiffness can change the nuclear membrane curvature, leading to stretching of the NPCs and reduced mechanical resistance allowing for increased nuclear import. Elosegui-Artola et al., 2017.

This effect, however, is dependent on the size of the molecular cargo as the effects of substrate stiffness were reduced as the molecular weight increased. This result suggests that stiffness can only enhance nuclear entry though the NPCs to a certain extent and that there is an optimal size range for these effects to be demonstrated. Because of this observation, it is possible that the increased cytoskeletal involvement from the hydrogels facilitate for quicker transport to the nucleus but is bottlenecked by the nuclear pore complex due to the rather large size of the Cas9 plasmid or RNP. Despite this finding we still see increasing editing efficiency on the hydrogels in a stiffness-dependent manner. What this observation suggests is that the true contributions of stiffness may lie within the nucleus during the gene editing process.

This suggestion led to an investigation of the nucleus itself. We posited that substrate stiffness may lead to changes in chromatin configuration, allowing for Cas9 to probe the DNA and cleave the target sequence more efficiently. To test this hypothesis, a commercial assay was used to test the chromatin accessibility of cells cultured on the 3 different hydrogel stiffnesses. Remarkably, it was at this stage that we were able to identify a potential reason behind the improvements in gene editing associated with stiffness. Based on the data, chromatin accessibility increases with stiffness and mimics the trends observed in all of the previous assays (FIG. 5E). This observation suggests that the predominant factor behind the additive effects of substrate stiffness is increased chromatin accessibility. This finding is further supported by the nucleus flattening as stiffness increases (FIG. 8). The effects of substrate stiffness are transmitted to the nucleus where they manifest as increased chromatin accessibility. As the assay itself assesses the chromatin accessibility in relation to a nuclease, it would be plausible then that the same principles would apply for the Cas9 nuclease.

In summary, our proposed mechanism is that substrate stiffness enhances in the CRISPR editing process via two processes. One of these processes involves the rapid transport of Cas9 to the nucleus via the cytoskeletal components, which are further recruited on hydrogel culture (FIG. 5F(i)). The other process is a result of increased cytoskeletal tension, which increases force transmission to the nucleus via the LINC complex and underlying nucleoskeleton. This result finally leads to increased chromatin accessibility and interactions with the Cas9 nuclease (FIG. 5F(ii)). The combination of these two processes brings us to our initial observations of increased editing on hydrogels in comparison to TCPS and increasing editing efficiency corresponding to substrate stiffness.

### 1.4.6 Outlook

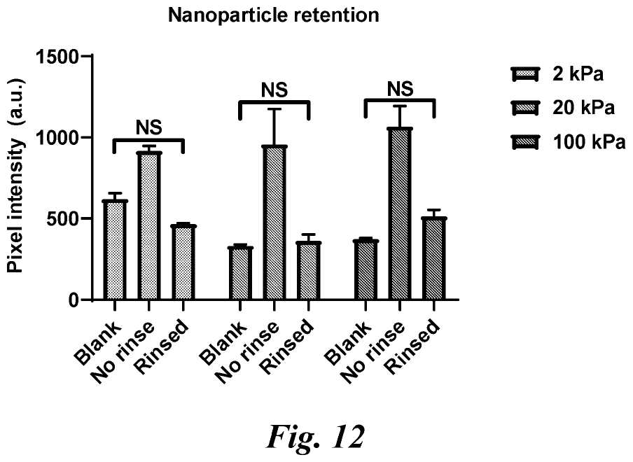

There were two primary objectives to this study. The first was to investigate how mechanical cues in the microenvironment, and more specifically stiffness could affect the gene editing efficiency. The second was to discover the mechanism by which this phenomenon occurs. It was found that gene editing was enhanced in cells cultured on hydrogels and that this improvement was dependent on the stiffness. Furthermore, additional experiments were performed to ensure these findings were not exclusive to any one hydrogel system, transfection platform, form of Cas9, gene target or cell line. These experiments effectively addressed many of the confounding factors that surrounded these findings. Through an extensive array of small molecules and siRNAs we find that the hydrogels enhance the gene editing through mechanobiology rather than acting as static reservoirs of the Cas9 cargo (FIG. 12).

When further insight was required, attention was focused on the nucleus. With a direct relation observed between chromatin accessibility and stiffness, we believe this finding is the most likely reason for the increased editing on hydrogels. The proposed mechanism is that substrate stiffness leads to increased cytoskeletal and nucleoskeletal involvement, which then manifests into increased chromatin accessibility. This increased accessibility would allow for the Cas9 RNP to better probe for the target DNA strand and initiate gene cleavage.

Our hope is that the findings of this Example will promote new avenues of research across various fields. The goal of many studies is to increase Cas9 editing efficiency by focusing on the delivery process or modifying the Cas9 cargo. Despite this large volume of work, there remains a question as to whether or not the microenvironment itself can be used to control the outcomes of gene editing. From the preceding data, it is apparent that mechanical cues are possible solutions. Future research could include further investigation into how other mechanical cues, such as surface chemistry and viscoelasticity, could impact gene editing or how these findings can be applied in vivo. The findings of this Example support the use mechanical cues in tandem with other methods for developing new CRISPR-Cas9 gene therapeutics for cell or gene therapy applications.

Based on the studies involving treatment with small molecules or biologics, it is evident that various components of mechano-transduction work in tandem with the canonical Cas9 gene editing process. This Example highlights the possibility of using small molecule drugs or therapeutics alongside Cas9 therapies to produce more favorable outcomes. It also is hopeful that this Example provides insightful information regarding the involvement of these components in gene editing previously unheard of. By confirming the involvement of these various components and their increasing involvement on hydrogels, we can move closer toward developing a more detailed mechanism as to how stiffness from the hydrogels enhances gene editing. Finally, what must also be considered is the nucleus and more specifically the nucleoskeleton. Though many studies focus on structural components in regard to the cytoskeleton, the nucleus has its own structural versions of these proteins, as well, and should be further investigated. Simon and Wilson, 2011.

### 1.5 Methods

### 1.5.1 Hydrogel Preparation

Sodium alginate rich in guluronic acid blocks and with a high molecular weight (212 kDa, Manugel DMB) was purchased from FMC BioPolymer. RGD coupling to the alginate backbone was achieved through carbodiimide chemistry. The concentration was such that 20 RGD peptides were coupled to one alginate chain. For a 2% (w/v) alginate hydrogel, the RGD concentration was 1,500 μM, as previously characterized. Rowley et al., 1999. Alginate was dialyzed in deionized water for 3 days using a dialysis membrane with a molecular weight cutoff of 3.5 kDa. After dialysis, the sample was treated with activated charcoal, sterile filtered, and lyophilized for 5 days. All samples were reconstituted in serum-free DMEM.

Polyacrylamide (PAM) hydrogel discs of different stiffnesses were generated by mixing different volumes of 40% (w/v) acrylamide, 2% (w/v) bis-acrylamide, and 1:100 and 1:1000 total volume of ammonium persulfate and tetramethylethylenediamine, respectively (Table 1). Immediately after mixing, the resultant solution was cast between glass plates separated by a 1-mm spacer. After 1 hour, the gels were punched into discs with a diameter of 15 mm and rinsed twice with DPBS(−) (Gibco) prior to storage at 4° C. for later use.

Collagen hydrogels were prepared with TeloCol-10 (Advanced BioMatrix) following the manufacturer's protocol. Gels were formed directly into well plates and equilibrated with DMEM 24 hours prior to seeding with cells. For increased stiffness, collagen hydrogels were treated with 20-mM genipin for 2 hours before rinsing 3 times with DMEM and left to equilibrate 24 hours prior to cell seeding. Linville et al., 2019. Acryloyl-PEG-RGD (Ac-PEG-RGD) was prepared as previously reported with slight modification. Burdick and Anseth, 2002.

In brief, GGGGRGDSP peptide was reacted with an equimolar amount of Ac-PEG-NHS (2,000 Da, Laysan Bio) in phosphate buffer (pH 8.0) overnight at room temperature. The reaction mixture was dialyzed against deionized water for 3 days (MWCO=3.5 kDa), sterile filtered, lyophilized, and stored at-30° C. Hydrogel disks (15 mm diameter and 1 mm thick before swelling) were fabricated with 5 wt %, 10 wt %, and 17.5 wt % PEGDA (575 Da, Sigma) in PBS, respectively, with the supplement of 4 mM Ac-PEG-RGD. The hydrogels were obtained within 45 min after the addition of ammonium persulphate (APS, 20 mM) and tetramethylethylenediamine (TEMED, 20 mM). The hydrogels were then equilibrated in PBS overnight prior to cell seeding.

### 1.5.2 Mechanical Characterization of Hydrogels

The MTS Criterion Model 43 mechanical testing system was used to measure the initial elastic modulus. All hydrogels were 2 mm in thickness and 15 mm in diameter. Gel discs were compressed to 15% strain at deformation rate of 1 mm min-1 according to a previous study. Chaudhuri et al., 2016. Within this regime, the stress-strain relation was linear, and the initial elastic modulus was calculated.

### 1.5.3 Cell Culture

U2OS.EGFP cells were kindly provided by Dr. J Keith Joung of Massachusetts General Hospital. Fu et al., 2013. These cells contain a single integrated copy of an EGFP-PEST gene. Reyon et al., 2012. Cells were cultured in DMEM (Gibco) with 10% FBS (Cytiva Life Sciences), 2 mM GlutaMAX (Gibco), 1% Penicillin-Streptomycin (10,000 U/mL, Gibco), and 400 μg/mL G418 (Gibco). For transfections, media without antibiotics was used to improve viability. Standard cell culture conditions of 37° C. and 5% CO₂ were employed for this study. Low passage human mesenchymal stem cells were kindly provided by the Food and Drug Administration (FDA).

### 1.5.4 2D Hydrogel Culture

12-mm Transwells® (Corning) without membrane inserts were mounted on top of the gels to ensure an even distribution of cells during seeding. Alginate hydrogels for 2D culture were prepared via ionic crosslinking as previously reported. Chaudhuri et al., 2015; Zhao et al., 2010.

The necessary amounts of RGD-modified alginate and calcium sulfate slurry (Table 1) were rapidly mixed using luer lock syringes (Cole-Parmer) and a female-female luer lock coupler (Value Plastics). The gel solution was quickly cast between 2 glass plates with a 1-mm spacer and allowed to gel for 45 minutes. 15-mm discs were then punched out and left to equilibrate in DMEM 24 hours prior to seeding of cells. Before seeding cells, gels were sterilized under UV for 30 minutes in the tissue culture hood. U2OS.EGFP cells were seeded at a density of 50,000 cells per cm².

PAM gels were equilibrated in DMEM 24 hours prior to functionalization of the surface with Collagen I (Corning). Tse and Engler, 2010. Briefly, the DMEM was removed and replaced with a 0.2-mg/mL solution of sulfo-SANPAH (Proteochem) diluted in 50-mM HEPES (pH 8.5) buffer and exposed to a 365-nm UV light source for 10 minutes. After rinsing twice with buffer, gels were coated with a 0.05-mg/mL solution of Collagen I and left to incubate overnight at 4° C. Prior to seeding cells, gels were rinsed twice with DPBS(−) and sterilized under UV for 30 minutes in the tissue culture hood. Cells were seeded at a density of 20,000 cells per cm² for U2OS.EGFP and 15,000 cells per cm² for human mesenchymal stem cells. Before seeding cells, PEG gels were sterilized under UV for 30 minutes in the tissue culture hood. U2OS.EGFP cells were seeded at a density of 40,000 cells per cm².

To create a more suitable tissue culture plastic control to serve as a comparison to the 2D hydrogels, a tissue culture-treated plastic coverslip (Sarstedt) was mounted onto a glass slide (1-mm thickness) with a small amount of cyanoacrylate adhesive. Cells were then seeded at the appropriate seeding density according to the cell types used.

### 1.5.5 Cell Spreading Measurements

ImageJ was used to measure the cell spreading on hydrogels 24 hours after seeding.

The polygon selection tool was used to draw an outline around the cell and the projected area was then calculated. For each image, 20-25 cells were analyzed. For all stiffnesses, three images were analyzed (N=3).

### 1.5.6 Cas9 Nanocomplex Preparation and Transfection

The pSpCas9 (PX165) plasmid was a gift from the Feng Zhang Lab (Addgene plasmid #48137). The GFP-T1 sgRNA plasmid was kindly provided by the George Church Lab (Addgene plasmid #41819).

Linear polyethylenimine (PEI) with a molecular weight of 25 kDa (Polysciences) was reconstituted in distilled water using 12.1-M hydrochloric acid to dissolve the polymer and 5-M sodium hydroxide to adjust the pH to 7.4. Final polymer concentration was 1 mg/mL. Lipofectamine™ 2000 (Invitrogen) also was included in this study as an additional transfection reagent.

All experiments were performed in non-treated 12 well plates (Falcon). Therefore, 0.5-ug total DNA was used per well for PAM and PEG 2D hydrogels. For experiments with alginate 2D hydrogels, cells were transfected with 3.0 μg of DNA. The spCas9:sgRNA plasmid weight ratio was maintained at 1:1. For transfections with PEI, a pDNA:PEI weight ratio of 1:2 was used. DNA and PEI solutions were prepared in individual 1.5-mL Protein LoBind® tubes (Eppendorf) prior to adding the PEI solution dropwise to the DNA solution and briefly mixed via vortex. The resultant polyplexes were spun down then left to incubate for 15 minutes then delivered to cell-laden hydrogels or coverslips. All samples were transfected overnight after which, the media was removed, and rinsed once with media to remove any residual complexes. After the addition of fresh cell culture media, samples were left to incubate for an additional 48 hours.

For studies with Lipofectamine™ 2000, a pDNA: Lipofectamine weight ratio of 1:1 was used, and transfections were conducted according to the manufacturer's protocol. All samples were rinsed after overnight treatment and left to incubate an additional 48 hours in the same manner as PEI transfection studies.

Cas9 mRNA was purchased from TriLink Biotechnologies (L-7206) and transfected with Lipofectamine™ MessengerMax™ (Invitrogen) following the manufacturer's protocol. The dosage used for all studies was 0.25 μg Cas9 mRNA and 0.025 μg sgRNA per well. All samples were transfected overnight prior to removal of media and rinsing with media. Analysis via flow cytometry was performed 24 hours later.

The Cas9 protein and EGFP-targeting sgRNA were purchased from Thermo Fisher (A36498, A35534) and transfected using Lipofectamine™ CRISPRMAX™ (Invitrogen) following the manufacturer's protocol. All samples were transfected overnight prior to removal of media and rinsing with media. Analysis via flow cytometry was performed 24 hours later.

### 1.5.7 Electroporation

For electroporation experiments, 1×10⁶ U2OS cells were resuspended in 200 μL of DPBS, transferred to a 2-mm electroporation cuvette (BioRad), mixed with 10 μg DNA, and electroporated using an Eppendorf Eporator (1 kV/cm). After electroporation, cells were immediately transferred to culture medium and incubated at room temperature for 5 mins. After centrifugation and resuspension in fresh culture medium, cells were used for seeding on well plates or hydrogels.

1.5.8 mCherry Expression Analysis

The pRZ-CAS9-mCherry plasmid was a gift from the Veit Hornung Lab (Addgene plasmid #80974). The total DNA content was fixed at 0.5 μg total DNA per well and the mCherry-Cas9:sgRNA plasmid ratio was maintained at 1:1. mCherry fluorescence was quantified using an EVOS M5000 Imaging System (Thermo Fisher Scientific). High magnification images for each condition were taken at 12, 24 and 48 hours and the percentage of mCherry-expressing cells were analyzed in ImageJ and calculated using the following equation:

$$\frac{Number of mCherry expressing cells}{Total number of cells} \times 100$$

For each experimental condition, 5 images were analyzed from 3 independent experiments. 1.5.9 Gel-nanoparticle interactions

PEI was conjugated with Texas Red™-X succinimidyl ester according to the manufactures' instructions. 1:1 dilution (TX Red-PEI:PEI) and phenol red free DMEM were used for this experiment. Nanocomplexes formed by TX Red-PEI and pDNA were added into each well containing hydrogels. After one day of incubation at 37° C., hydrogels were washed twice with phenol red free DMEM and mounted on #1.7 coverslip for imaging. Multiple images were taken for each of the conditions using a laser scanning confocal microscope (Zeiss, LSM 800). Pixel intensity for each image were quantified by ImageJ for analysis.

### 1.5.10 Plasmid Uptake

pDNA was tagged with Cy5 using Label IT® nucleic acid labeling kit according to the manufacturer's instructions. 1:2 dilution (Cy5-pDNA: pDNA) were used for all experiments. For cellular uptake experiment, diluted Cy5-pDNA was mixed with PEI to form nanoparticles and then used for transfection. After incubation with cells for 4 h, 12 h, and 22h, nanoparticles in medium were removed by aspiration and washing for two times. Cells were detached from the substrate and pelleted by centrifugation. After resuspending in DPBS containing 2% FBS, cell samples were analyzed by flow cytometry.

### 1.5.11 EGFP Gene Disruption

A BD FACSCanto Flow Cytometer (BD Biosciences) was used to quantify the EGFP negative cell population in all samples. For plasmid Cas9 studies, analysis was performed 48 hours after overnight transfection. For Cas9 mRNA and RNP studies, analysis was performed 24 hours after overnight transfection.

To retrieve cells from collagen-coated PAM 2D gels, samples were first rinsed with DPBS(−). Collagenase/Dispase (Roche, 1 mg/mL) was then added, and samples were left to incubate at 37° C. under gentle shaking for 10 minutes. The collected cells were then spun down in a centrifuge at 300×g for 5 minutes. The supernatant was discarded then samples were resuspended in a 2% FBS solution in DPBS(−).

For alginate hydrogels, a previously described method was employed. Darnell et al., 2018. Gels were placed into 5 mL Eppendorf tubes and incubated with 50 mM EDTA in HEPES for 10 minutes. Trypsin (0.25%) was then added to ensure cell detachment from alginate chains and samples were incubated for an additional 5 minutes at 37° C. Cells were then centrifuged at 300×g for 5 minutes before proceeding with further analysis.

PEG hydrogels were first rinsed with DPBS(−) then incubated with Trypsin (0.25%) for 5 minutes under gentle shaking. Equal volume of cell culture media was added to neutralize the reaction. Samples were spun down at 300×g for 5 minutes then resuspended in 2% FBS in DPBS(−).

### 1.5.12 Small Molecule Inhibitors

The small molecule inhibitors, Y-27632 (10 μM, MedChemExpress), Cytochalasin-D (1 μM, APExBio), ML 7 hydrochloride (25 μM, MedChemExpress), and Verteporfin (1 μM, MedChemExpress) were used to inhibit common components of various mechano-transduction pathways. For these studies, samples were treated with the inhibitors for 4 hours prior to transfection. Wang et al., 2017. For activation of the Rho pathway, lysophosphatidic acid (Sigma-Aldrich, L7260) was used at concentrations of 5, 10 and 20 μM. Prior to treatment, all samples were serum starved for 1 hour. Amerongen et al., 2000. Lysophosphatidic acid at the above concentrations were then added and samples were left to incubate for 4 hours before transfection. 1.5.13 siRNA inhibition siRNA studies were performed to study the contributions of various constituents of the LINC complex on gene editing. All siRNA sequences used for this study were purchased from Horizon. The ON-TARGETplus and SMARTpool modifications were chosen to ensure gene silencing. Transfections were performed with Lipofectamine™ 2000 at a siRNA: lipid ratio of 20:1 (pmol: volume). For all siRNA studies, 12.5 μmol siRNA was used and treated for 24 hours. Afterwards, media was removed, and samples were rinsed with culture media prior to transfection with PEI-Cas9 nanocomplexes overnight. The following day, media was again removed, and samples were rinsed prior to replacement with fresh culture media. Samples were then left to incubate for an additional 48 hours.

### 1.5.14 Endogenous Gene Disruption

U2OS.EGFP cells or MSCs were transfected with the Cas9 plasmid and sgRNA plasmid targeting AAVS1. The GeneArt™ Genomic Cleavage Detection Kit (Thermo Fisher) was then used to semi-quantitatively assess insertion and deletion (Indel) formation. Forward and reverse primers for the AAVS1 gene were purchased from Thermo Fisher.

### 1.5.15 Gene Editing of Hard-to-Transfect Cell Line

Low passage mesenchymal stem cells were seeded onto collagen-coated PAM gels or TC coverslips at a density of 15,000 cells per cm² and left to adhere overnight. Cas9 mRNA and sgRNA were used at dosages of 0.5 μg Cas9 mRNA and 0.05 μg sgRNA respectively per well.

### 1.5.16 Sanger Sequencing

Sanger sequencing was performed by The Genetic Resources Core Facility at The Johns Hopkins University, School of Medicine (RRID:SCR_018669). Cell lysis and PCR steps were done according to the protocols included in the GeneArt™ Genomic Cleavage Detection Kit. After verification of the PCR product using gel electrophoresis, samples were purified using the PureLink™ PCR Purification Kit (Invitrogen). For each sample separate forward and reverse sequencing reactions were performed using the PCR primers for AAVS1.

The TIDE (Tracking of Indels by Decomposition) software package was used quantitatively assess the extent of gene editing among the various conditions. Chromatograms for untreated cells cultured on tissue culture polystyrene cover slips were used as reference points for analysis. Brinkman et al., 2014.

### 1.5.17 Nuclear Extraction for Flow Cytometry

For nuclear transport experiment, U2OS cells were electroporated with Cy5-pDNA as described above. After one day of culture on substrate, cell nuclear extract was performed according to a previous method with some alterations. Baslan et al., 2012. Cells are detached from the culture substrate and pelleted by centrifugation. Then NST-DAPI buffer supplemented with 5 mM EDTA was then added to resuspend the pellet. Tubes are incubated on wet ice for 30 mins. The cell suspension was then centrifuged at 1,000×g for 5 mins to collect nuclei pellet. After resuspending in DPBS containing 2% FBS, nuclei samples were ready for flow cytometry.

### 1.5.18 Chromatin Accessibility

Chromatin accessibility was assessed using the EpiQuik Chromatin Accessibility Assay Kit (EpiGentek). Cells were seeded on hydrogels or TCPS at a density of 20,000 cells/cm². Culture media was replaced the following day and samples were left in culture for an additional 24 hours. Cells were then collected prior to proceeding with the assay in accordance with the manufacturer's protocols.

### Example 2

### Fabrication of Polyacrylamide (PAM) 2D Hydrogels

### 2.1 Materials

40 wt % (w/v) acrylamide stock solution was resuspended in PBS and sterilized. 2 wt % (w/v) bis-acrylamide stock solution was resuspended in PBS and sterilized. 10 wt % (w/v) ammonium persulfate (APS) was resuspended in distilled H₂O. Other reagents include tetramethylethylenediamine (TEMED), distilled H₂O, and PBS (water also has been used for this procedure. A 15-mm biopsy punch (or desired size), Sigmacote® (a solution of a chlorinated organopolysiloxane in heptane that readily forms a covalent, microscopically thin film on glass), 25-x 75-mm glass slides with 1-mm thickness, glass plates, and 15-mL conical tubes (any type of tube can be used dependent on volume).

### 2.2 Representative Formulations

Representative formulations are provided in Table 1.

TABLE 1
Representative Formulations

| | | Final total | | | | | | |
|---|---|---|---|---|---|---|---|---|
| Young's | Total | acrylamide | Acrylamide | Final total bis- | Bis-acrylamide | Ammonium | | |
| Modulus | Volume | content | stock solution | acrylamide content | stock solution to | persulfate | TEMED | Water or |
| (kPa) | (mL) | wt % (w/v) | to add (mL) | wt % (w/v) | add (mL) | (mL) | (mL) | PBS (mL) |
| 2 | 5 | 10 | 1.25 | 0.020 | 0.05 | 0.05 | 0.005 | 3.70 |
| 20 | 5 | 10 | 1.25 | 0.18 | 0.45 | 0.05 | 0.005 | 3.30 |
| 100 | 5 | 20 | 2.5 | 0.5 | 1.25 | 0.05 | 0.005 | 1.25 |

### Example 3

Functionalization of PAM Hydrogels with Extracellular Matrix Proteins (ECM)

### 3.1 Materials

PAM 2D gels were generated using the protocol provided in Example 2. Reagents and materials include Sulfo-SANPAH (c1111-100 mg) (available from proteochem.com/sulfosanpahcrosslinker100 mg-p-102.html), 50-mM HEPES buffer pH 8.5, sterilized, DMEM, and ECM, e.g., fibronectin, collagen 1, and the like, and PBS. In particular embodiments, collagen 1 is used as the ECM protein. A 365-nm UV light source, e.g., a nail lamp is suitable for use with this method because it fit a well plate.

### 3.2 Surface Functionalization and ECM Tethering

Prior to coating, equilibrate the gels in DMEM overnight. On the day of coating, remove the DMEM and rinse with 50-mM HEPES buffer pH 8.5. Weigh and dissolve Sulfo-SANPAH at a concentration of 0.2 mg/mL in the HEPES buffer. The Sulfo-SANPAH solution is best prepared fresh and stock solutions should not be made as it is light, temperature, and moisture-sensitive. Minimize exposure to air and work as quickly as possible. Add enough (0.5 mL-3 mL) Sulfo-SANPAH solution to cover the surface of the gels and expose to 365-nm UV for 10 minutes. Rinse twice with buffer to remove excess Sulfo-SANPAH. Prepare the ECM solution in buffer at a concentration of your choice. As some ECM proteins may precipitate, it is important to vortex vigorously at this step and prior to coating the gels. Collagen 1 was used at a concentration of 0.05 mg/mL. Concentrations ranging from 0.025-0.10 mg/mL have been used. Fibronectin also has been used at concentrations ranging from 10-20 μg/μL. Add the ECM solution to gels and incubate overnight at 4° C. if desired. For these studies 4° C. was used to prevent excessive gelation onto the surface of the gel. This reaction has been carried out in temperatures of 4, 25 and 37° C. all overnight.

### Example 4

### Seeding of U2OS Cells onto ECM-Coated PAM Hydrogels

### 4.1 Materials

ECM-coated PAM 2D gels were prepared as described in Example 3. Materials and reagents include cells (for these studies a U2OS.EGFP cell line was used), PBS, Trypsin-EDTA (0.05%), phenol red, and 12-mm Transwell® with 0.4-μm Pore Polycarbonate Membrane Insert, Sterile (Corning, Product Number: 3401) (to ensure even seeding onto the gel and modified for these experiments by removing the insert).

Other materials include cell culture media without antibiotics. For transfection studies, the media consisted of: DMEM, high glucose, pyruvate, 450 mL (Thermo Fisher Scientific, Catalog Number: 11995040), HyClone Characterized Fetal Bovine Serum (FBS), U.S. Origin, 50 mL (Cytiva, Catalog Number: SH30071.03HI), GlutaMAX™ Supplement, 2 mM final concentration (Thermo Fisher Scientific, (Catalog Number: 35050061)

4.2 Seeding Cells onto ECM-Coated Hydrogels

Ensure that cells are of proper confluence and passage number prior to experiments. Cells of 70-90% confluency were used. Passage numbers below 35 were used to reduce likelihood of excessive genomic instability. Cells were cultured in a T-75 flask.

Aspirate media and rinse with 10 mL of PBS. Aspirate the PBS and add 2 mL of Trypsin and incubate for 3 minutes at 37° C. Neutralize the reaction with equal volume media and spin down at 300×g for 5 minutes. Aspirate and resuspend in media without antibiotics. Count cells using a suitable method known in the art.

Prior to seeding, aspirate the PBS from the sterilized hydrogels and carefully place an hollow transwell (no membrane) or customized holder on top of the gels. Add 1.5 mL of media outside the hollow transwell and 0.4 mL inside. Add the desired number of cells in 100 μL of media into the transwell. Carefully pipet up and down several times to allow for even cell seeding. For U2OS cells, a density of 180-1000 cells/μL were tested. The range of 180-200 cells/μL is a representative seeding density. Incubate cells overnight at 37° C. prior to transfection.

### Example 5

### Transfection of PAM 2D Hydrogels with CRISPR-Cas9 Cargo

### 5.1 Materials

Cell-seeded hydrogels are prepared as provided in Example 4. Reagents and materials include DMEM, high glucose, pyruvate, (Thermo Fisher Scientific, Catalog Number: 11995040), Cas9 Plasmid (Addgene, Plasmid #48137), GFP-T1 sgRNA Plasmid (Addgene, Plasmid #41819), and polyethylenimine, linear, MW 25000 (Polysciences, Catalog Number: 23966-100) aliquoted at a concentration of 1 mg/mL. While linear PEI was the primary transfection reagent used for these studies, Lipofectamine™ 2000 Transfection Reagent also was used (Thermo Fisher Scientific, Catalog Number: 11668019) according to manufacturer's protocol. Protein LoBind Tubes, Protein LoBind®, 1.5 mL, PCR clean (Eppendorf, Catalog Number: 0030108442) also were used.

### 5.2 Transfection of PAM 2D Hydrogels

Prepare one tube for the DNA solution and one tube for the PEI solution. For each well, there should be about 25 μL of DNA solution at 0.5 μg/well and 25 μL of 1.0 μg/well linear PEI for a combined volume of 50 μL nanoparticle solution per well. For these studies plasmid amount ranging from 0.25-1.0 μg was used with 0.5 μg giving the optimal results. Final nanoparticle volumes of 50-300 μL also have been tested.

Calculate the necessary amount of DNA for n+1 wells and PEI for n+2 wells. This calculation will account for pipetting error. Slowly, add equal volume of PEI solution to the DNA mixture and mix for 5 seconds via vortexing. We also have tested mixing via pipetting. Incubate for 15 minutes. Incubation times of 15-20 minutes should be sufficient. Carefully aspirate the media from the cell-seeded hydrogels and add 1 mL of fresh media. Gently mix the nanoparticle solution and pipet the nanoparticle solution into the well. Gently pipet up and down to ensure even distribution of nanoparticles within the well. Incubate overnight. Remove the media and rinse with 1 mL of media. This step ensures that no residual nanoparticles remain. Add 1 mL of media into each well and continue experiment for 48 hours. At the end of the experiment, analyze the gene editing efficiency using a validated method, such as flow cytometry, image analysis, sequencing or a commercial genome cleavage detection assay.

### Example 6

### Matrix Stiffness Regulates the Homology-Directed Repair (HDR) of CRISPR-Cas9 Gene Editing and Hydrogels with Controlled Stiffness Enhances HDR

### 6.1 Materials and Methods: Homology-Directed Repair

### 6.1.1 Hydrogel Preparation

Polyacrylamide (PAM) hydrogel discs of different stiffnesses were generated by mixing different volumes of 40% (w/v) acrylamide, 2% (w/v) bis-acrylamide, and 1:100 and 1:1000 total volume of ammonium persulfate and tetramethylethylenediamine, respectively. Immediately after mixing, the resultant solution was immediately cast between glass plates separated by a 1-mm spacer. After 1 hour, the gels were punched into discs with a diameter of 15 mm and rinsed twice with DPBS(−) (Gibco) prior to storage at 4° C. for later use.

### 6.1.2 Cas9 RNP Nanocomplex Formation

The Cas9 protein and EGFP-targeting sgRNA were purchased from Thermo Fisher (A36498, A35534) and transfected using Lipofectamine CRISPRMAX (Invitrogen) following the manufacturer's protocol. A dosage of 0.5-ug Cas9 protein, 125-ng sgRNA, 0.5-μg ssODN donor template and 1.5-μL Lipofectamine CRISPRMAX was chosen for a 12 well plate format with 1 mL of total medium per well. All samples were transfected overnight prior to removal of media and rinsing with media. Analysis via Sanger sequencing was performed 48 hours later.

### 6.1.3 Cell Seeding and Small Molecule Inhibitor Treatment

For cell seedings, 12-mm Transwells® (Corning) transwells without membrane inserts were mounted on top of the gels to ensure an even distribution of cells during seeding. A seeding density of 10,000 cells/cm² was used for all studies. If small molecules were used for enhancing HDR, cells were incubated with nocodazole at a dosage of 20 ng/mL for 24 hours then rinsed with culture media prior to transfection.

### 6.1.4 Sanger Sequencing

Sanger sequencing was performed by The Genetic Resources Core Facility at The Johns Hopkins University, School of Medicine (RRID: SCR_018669). Cell lysis and PCR steps were done according to the protocols included in the GeneArt™ Genomic Cleavage Detection Kit. After verification of the PCR product using gel electrophoresis, samples were purified using the PureLink™ PCR Purification Kit (Invitrogen). For each sample separate forward and reverse sequencing reactions were performed using the PCR primers for EGFP.

The Synthego ICE tool was used to analyze Sanger sequencing traces. The Knock-in (KI) score was used as a measure of HDR efficiency.

### 6.2 Results

As shown in FIG. 13, substrate stiffness regulates the Homology-Directed Repair (HDR) of CRISPR-Cas9 gene editing. In addition, a significantly enhanced HDR is achieved when using hydrogels with optimized stiffness for cell culture and gene editing as compared to using conventional tissue culture plastic (TCP). FIG. 13.

### Example 7

### Hydrogels with Controlled Stiffness can Enhance CRISPR-Cas9 Gene Editing of T Cells for Potential Adoptive T Cell Therapies

Adoptive cell therapies (ACT) provide an exciting new approach for the treatment of cancers. Currently, a number of strategies for the generation of adoptive T cell therapies are being studied in the clinic, including isolation and expansion of tumor infiltrating lymphocytes (TILs), engineered T-cell receptors for cancer targeting (TCR), and chimeric antigen receptors (CARs) for extracellular targeting of cancer antigens. CRISPR-Cas9 gene editing is currently widely used in clinical trials to enhance these therapies.

### 7.1 Materials and Methods: CRISPR-Cas9 Gene Editing of T Cells for Potential Adoptive T Cell Therapies

### 7.1.1 Hydrogel Preparation

Polyacrylamide (PAM) hydrogel discs of different stiffnesses were generated by mixing different volumes of 40% (w/v) acrylamide, 2% (w/v) bis-acrylamide, and 1:100 and 1:1000 total volume of ammonium persulfate and tetramethylethylenediamine, respectively. Immediately after mixing, the resultant solution was immediately cast between glass plates separated by a 1-mm spacer. After 1 hour, the gels were punched into discs with a diameter of 15 mm and rinsed twice with DPBS(−) (Gibco) prior to storage at 4° C. for later use.

### 7.1.2 T Cell Purification

CD4 and CD8 T cells were isolated from PBMCs by negative selection using STEMCELL EasySep isolation kits (STEMCELL Technologies, 17952 and 17953), and mixed 1:1. T cells were then activated with ImmunoCult™ Human CD3/CD28 T Cell Activator (STEMCELL Technologies, 10971), for 72 hours with 100-IU/mL IL2 (Peprotech, 200-02) in X-Vivo 15 media (Lonza, 02-053Q) supplemented with 5% human AB serum (Millipore Sigma, H4522), 2-mM L-glutamine (Gibco, 25030081), and 100-U/mL penicillin and 100-mg/mL streptomycin (Gibco, 15140122). T cells were then expanded for 9-12 days in media and 100-IU/mL IL2 before use in experiments or storage in liquid nitrogen.

### 7.1.3 Cas9 RNP Nanocomplex Formation

The Cas9 protein and AAVS1-targeting sgRNA were purchased from Thermo Fisher (A36498, A35534) and transfected using Lipofectamine CRISPRMAX (Invitrogen) following the manufacturer's protocol. A dosage of 2.5-μg Cas9 protein, 15-pmol sgRNA, and 3.0-μL Lipofectamine CRISPRMAX was chosen for a 12-well plate format with 1 mL of total medium per well. All samples were transfected overnight prior to removal of media and rinsing with media. Analysis via Sanger sequencing was performed 48 hours later.

### 7.1.4 Cas9 Electroporation of CD3+ T Cells

For optimum results, isolated T cells were activated for 72 hours prior to electroporation. Cells were collected, rinsed, then resuspended in Opti-MEM™ with GlutaMAX™ (Gibco, 51985034). A final volume of 50 μL consisting of 500,000 cells, 7.2-ug Cas9 and 240-pmol sgRNA was placed in a 2-mm electroporation cuvette (BioRad), and electroporated using an Eppendorf Eporator (1 kV/cm). Samples were placed into culture medium immediately after electroporation. After centrifugation and resuspension, cells were seeded onto hydrogels or well plates at a density of 50,000 cells/cm².

For cell seedings, 12-mm Transwells® (Corning) transwells without membrane inserts were mounted on top of the gels to ensure an even distribution of cells during seeding.

### 7.1.5 Flow Cytometry

200,000 CD3+ T cells were collected 3 days after activation and stained for PE anti-CD49a (1:200), PE/Cy7 anti-CD4 antibody (1:200), APC anti-CD8 antibody (1:200), and PerCP anti-CD25 (1:200) and Zombie NIR™ Fixable Viability Kit (1:1000).

### 7.1.6 Sanger Sequencing

Sanger sequencing was performed by The Genetic Resources Core Facility at The Johns Hopkins University, School of Medicine (RRID: SCR_018669). Cell lysis and PCR steps were done according to the protocols included in the GeneArt™ Genomic Cleavage Detection Kit. After verification of the PCR product using gel electrophoresis, samples were purified using the PureLink™ PCR Purification Kit (Invitrogen). For each sample separate forward and reverse sequencing reactions were performed using the PCR primers for AAVS1.

The TIDE (Tracking of Indels by Decomposition) software package was used to quantitatively assess the extent of gene editing among the various conditions. Chromatograms for untreated cells cultured on tissue culture polystyrene cover slips were used as reference points for analysis.

### 7.2 Results

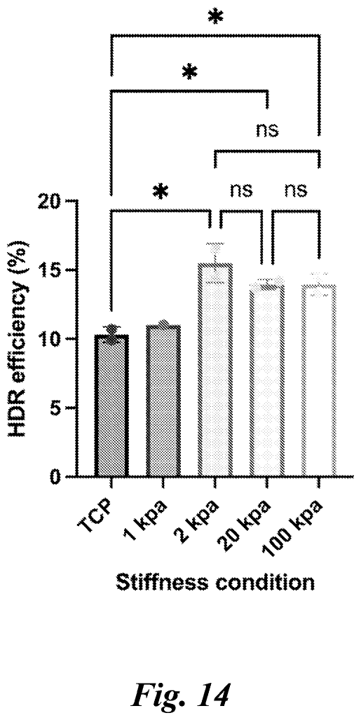

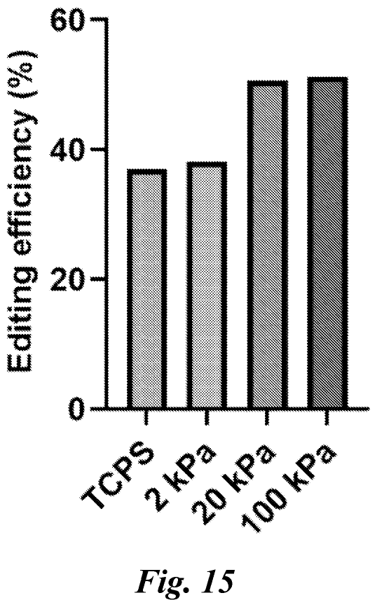

As shown in FIG. 14, Our new data show that hydrogels with optimal stiffness can provide mechanical stimulations that will enhance T cell gene editing.

Activated T cells were resuspended in Opti-MEM with GlutaMAX, and electroporation was used to transfect the cells with Cas9 protein and sgRNA targeting the AAVS1 gene. T-cells were then cultured on hydrogels with different stiffness or on TCPS after electroporation. Our data shows that 24 hours after electroporation, there is a clear effect of substrate stiffness on T cell gene editing efficiency. The 20- and 100-kPa conditions demonstrate the optimum gene editing and show enhanced editing efficiency as compared to the TCPS and 2-kPa conditions (FIG. 14).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Eoh, J. and Gu, L. Biomaterials as vectors for the delivery of CRISPR-Cas9. Biomater. Sci. 7, 1240-1261 (2019).
Glass, Z., Lee, M., Li, Y. and Xu, Q. Engineering the Delivery System for CRISPR-Based Genome Editing. Trends Biotechnol. 36, 173-185 (2018).
Doudna, J. A. and Charpentier, E. The new frontier of genome engineering with CRISPR-Cas9. Science 346, 1258096 (2014).
Sternberg, S. H., Redding, S., Jinek, M., Greene, E. C. and Doudna, J. A. DNA interrogation by the CRISPR RNA-guided endonuclease Cas9. Nature 507, 62-67 (2014).
Ondrej, V., Lukásová, E., Falk, M. and Kozubek, S. The role of actin and microtubule networks in plasmid DNA intracellular trafficking. Acta Biochim. Pol. 54, 657-663 (2007).
Coppola, S. et al. The role of cytoskeleton networks on lipid-mediated delivery of DNA. Ther. Deliv. 4, 191-202 (2013).
Modaresi, S., Pacelli, S., Whitlow, J. and Paul, A. Deciphering the role of substrate stiffness in enhancing the internalization efficiency of plasmid DNA in stem cells using lipid-based nanocarriers. Nanoscale 10, 8947-8952 (2018).
Persson, M. et al. Transportation of Nanoscale Cargoes by Myosin Propelled Actin Filaments. PLOS ONE 8, e55931 (2013).
Cho, S., Irianto, J. and Discher, D. E. Mechanosensing by the nucleus: From pathways to scaling relationships. J. Cell Biol. 216, 305-315 (2017).
Huang, C. et al. Substrate Stiffness Regulates Cellular Uptake of Nanoparticles. Nano Lett. 13, 1611-1615 (2013).
García-García, M. et al. Mechanical control of nuclear import by Importin-7 is regulated by its dominant cargo YAP. Nat. Commun. 13, 1174 (2022).
Tajik, A. et al. Transcription upregulation via force-induced direct stretching of chromatin. Nat. Mater. 15, 1287-1296 (2016).
Caiazzo, M. et al. Defined three-dimensional microenvironments boost induction of pluripotency. Nat. Mater. 15, 344-352 (2016).
Jain, N., Iyer, K. V., Kumar, A. and Shivashankar, G. V. Cell geometric constraints induce modular gene-expression patterns via redistribution of HDAC3 regulated by actomyosin contractility. Proc. Natl. Acad. Sci. 110, 11349-11354 (2013).
Le, H. Q. et al. Mechanical regulation of transcription controls Polycomb-mediated gene silencing during lineage commitment. Nat. Cell Biol. 18, 864-875 (2016).
Ventre, M. and Netti, P. A. Engineering Cell Instructive Materials To Control Cell Fate and Functions through Material Cues and Surface Patterning. ACS Appl. Mater. Interfaces 8, 14896-14908 (2016).
Rosazza, C. et al. Endocytosis and Endosomal Trafficking of DNA After Gene Electrotransfer In Vitro. Mol. Ther.-Nucleic Acids 5, e286 (2016).
Li, L., Eyckmans, J. and Chen, C. S. Designer biomaterials for mechanobiology. Nat. Mater. 16, 1164-1168 (2017).
Lin, Y., Wagner, E. and Lächelt, U. Non-viral delivery of the CRISPR/Cas system: DNA versus RNA versus RNP. Biomater. Sci. 10, 1166-1192 (2022).
Xu, C.-F. et al. Rational designs of in vivo CRISPR-Cas delivery systems. Deliv. Biomacromolecules Ther. Genome Ed. 168, 3-29 (2021).
Behr, M., Zhou, J., Xu, B. and Zhang, H. In vivo delivery of CRISPR-Cas9 therapeutics: Progress and challenges. Hot Top. Rev. Drug Deliv. 11, 2150-2171 (2021).
Tong, S., Moyo, B., Lee, C. M., Leong, K. and Bao, G. Engineered materials for in vivo delivery of genome-editing machinery. Nat. Rev. Mater. 4, 726-737 (2019).
Brinkman, E. K., Chen, T., Amendola, M. and van Steensel, B. Easy quantitative assessment of genome editing by sequence trace decomposition. Nucleic Acids Res. 42, e168 (2014).
Sansbury, B. M., Hewes, A. M. and Kmiec, E. B. Understanding the diversity of genetic outcomes from CRISPR-Cas generated homology-directed repair. Commun. Biol. 2, 458 (2019).
Javaid, N. and Choi, S. CRISPR/Cas System and Factors Affecting Its Precision and Efficiency. Front. Cell Dev. Biol. 9, (2021).
Wang, T., Wei, J. J., Sabatini, D. M. and Lander, E. S. Genetic Screens in Human Cells Using the CRISPR-Cas9 System. Science 343, 80-84 (2014).
Guo, T. et al. Harnessing accurate non-homologous end joining for efficient precise deletion in CRISPR/Cas9-mediated genome editing. Genome Biol. 19, 170 (2018).
Bermudez-Cabrera, H. C. et al. Small molecule inhibition of ATM kinase increases CRISPR-Cas9 1-bp insertion frequency. Nat. Commun. 12, 5111 (2021).
de Carvalho, T. G. et al. A simple protocol for transfecting human mesenchymal stem cells. Biotechnol. Lett. 40, 617-622 (2018)
Hamann, A., Nguyen, A. and Pannier, A. K. Nucleic acid delivery to mesenchymal stem cells: a review of nonviral methods and applications. J. Biol. Eng. 13, 7 (2019).
Manotham, K. and Chattong, S. Genome Editing of MSCs as a Platform for Cell Therapy. in Zinc Finger Proteins: Methods and Protocols (ed. Liu, J.) 125-138 (Springer New York, 2018). doi:10.1007/978-1-4939-8799-3_10.
Golchin, A., Shams, F. and Karami, F. Advancing Mesenchymal Stem Cell Therapy with CRISPR/Cas9 for Clinical Trial Studies. in Cell Biology and Translational Medicine, Volume 8: Stem Cells in Regenerative Medicine (ed. Turksen, K.) 89-100 (Springer International Publishing, 2020). doi:10.1007/5584_2019 459.
Hendel, A. et al. Chemically modified guide RNAs enhance CRISPR-Cas genome editing in human primary cells. Nat. Biotechnol. 33, 985-989 (2015).
Janmey, P. A., Fletcher, D. A. and Reinhart-King, C. A. Stiffness Sensing by Cells. Physiol. Rev. 100, 695-724 (2020).
Shi, J. et al. Myosin light chain kinase-independent inhibition by ML-9 of murine TRPC6 channels expressed in HEK293 cells. Br. J. Pharmacol. 152, 122-131 (2007).
Cai, S. et al. Regulation of cytoskeletal mechanics and cell growth by myosin light chain phosphorylation. Am. J. Physiol.-Cell Physiol. 275, C1349-C1356 (1998).
Chen, C. et al. Myosin Light Chain Kinase (MLCK) Regulates Cell Migration in a Myosin Regulatory Light Chain Phosphorylation-independent Mechanism*. J. Biol. Chem. 289, 28478-28488 (2014).
Ñeco, P. et al. New Roles of Myosin II during Vesicle Transport and Fusion in Chromaffin Cells*. J. Biol. Chem. 279, 27450-27457 (2004).
Cho, S., Irianto, J. and Discher, D. E. Mechanosensing by the nucleus: From pathways to scaling relationships. J. Cell Biol. 216, 305-315 (2017).
Grady, M. E. et al. Intracellular nanoparticle dynamics affected by cytoskeletal integrity. Soft Matter 13, 1873-1880 (2017).
Snider, J. et al. Intracellular actin-based transport: How far you go depends on how often you switch. Proc. Natl. Acad. Sci. 101, 13204-13209 (2004).
Gupta, M. et al. Adaptive rheology and ordering of cell cytoskeleton govern matrix rigidity sensing. Nat. Commun. 6, 7525 (2015).
Kechagia, J. Z., Ivaska, J. and Roca-Cusachs, P. Integrins as biomechanical sensors of the microenvironment. Nat. Rev. Mol. Cell Biol. 20, 457-473 (2019).
Caliari, S. R. and Burdick, J. A. A practical guide to hydrogels for cell culture. Nat. Methods 13, 405-414 (2016).
Khaitlina, S. Yu. Intracellular transport based on actin polymerization. Biochem. Mosc. 79, 917-927 (2014).
Lieleg, O., Claessens, M. M. A. E. and Bausch, A. R. Structure and dynamics of cross-linked actin networks. Soft Matter 6, 218-225 (2010).
Ishizaki, T. et al. Pharmacological Properties of Y-27632, a Specific Inhibitor of Rho-Associated Kinases. Mol. Pharmacol. 57, 976 (2000).
Hoon, J. L. A.-T., Mei H. AU-Koh, Cheng-GeeTI-The Regulation of Cellular Responses to Mechanical Cues by Rho GTPases. Cells 5, (2016).
Amerongen, G. P. van N., Vermeer, M. A. and van Hinsbergh, V. W. M. Role of RhoA and Rho Kinase in Lysophosphatidic Acid-Induced Endothelial Barrier Dysfunction. Arterioscler. Thromb. Vasc. Biol. 20, e127-e133 (2000).
Panciera, T., Azzolin, L., Cordenonsi, M. and Piccolo, S. Mechanobiology of YAP and TAZ in physiology and disease. Nat. Rev. Mol. Cell Biol. 18, 758-770 (2017).
Wang, C. et al. Verteporfin inhibits YAP function through up-regulating 14-3-30 sequestering YAP in the cytoplasm. Am. J. Cancer Res. 6, 27 (2016).
Kurotsu, S. et al. Soft Matrix Promotes Cardiac Reprogramming via Inhibition of YAP/TAZ and Suppression of Fibroblast Signatures. Stem Cell Rep. 15, 612-628 (2020).
Uhler, C. and Shivashankar, G. V. Regulation of genome organization and gene expression by nuclear mechano-transduction. Nat. Rev. Mol. Cell Biol. 18, 717-727 (2017).
Zhang, Q. et al. Nesprin-1 and -2 are involved in the pathogenesis of Emery-Dreifuss muscular dystrophy and are critical for nuclear envelope integrity. Hum. Mol. Genet. 16, 2816-2833 (2007).
Lammerding, J. et al. Lamins A and C but Not Lamin B1 Regulate Nuclear Mechanics*. J. Biol. Chem. 281, 25768-25780 (2006).
Lerman, M. J., Lembong, J., Muramoto, S., Gillen, G. and Fisher, J. P. The evolution of polystyrene as a cell culture material. Tissue Eng. Part B Rev. 24, 359-372 (2018).
Simon, D. N. and Wilson, K. L. The nucleoskeleton as a genome-associated dynamic ‘network of networks’. Nat. Rev. Mol. Cell Biol. 12, 695-708 (2011).
Burke, B. and Stewart, C. L. The nuclear lamins: flexibility in function. Nat. Rev. Mol. Cell Biol. 14, 13-24 (2013).
Li, P. and Noegel, A. A. Inner nuclear envelope protein SUNI plays a prominent role in mammalian mRNA export. Nucleic Acids Res. 43, 9874-9888 (2015).
Liu, Q. et al. Functional association of Sun1 with nuclear pore complexes. J. Cell Biol. 178, 785-798 (2007).
Bai, H., Lester, G. M. S., Petishnok, L. C. and Dean, D. A. Cytoplasmic transport and nuclear import of plasmid DNA. Biosci. Rep. 37, BSR20160616 (2017).
Elosegui-Artola, A. et al. Force Triggers YAP Nuclear Entry by Regulating Transport across Nuclear Pores. Cell 171, 1397-1410.e14 (2017).
Rowley, J. A., Madlambayan, G. and Mooney, D. J. Alginate hydrogels as synthetic extracellular matrix materials. Biomaterials 20, 45-53 (1999).
Linville, R. M. et al. Human iPSC-derived blood-brain barrier microvessels: validation of barrier function and endothelial cell behavior. Biomaterials 190-191, 24-37 (2019).
Burdick, J. A. and Anseth, K. S. Photoencapsulation of osteoblasts in injectable RGD-modified PEG hydrogels for bone tissue engineering. Inject. Polym. Biomater. 23, 4315-4323 (2002).
Chaudhuri, O. et al. Hydrogels with tunable stress relaxation regulate stem cell fate and activity. Nat. Mater. 15, 326-334 (2016).
Fu, Y. et al. High-frequency off-target mutagenesis induced by CRISPR-Cas nucleases in human cells. Nat. Biotechnol. 31, 822-826 (2013).
Reyon, D. et al. FLASH assembly of TALENs for high-throughput genome editing. Nat. Biotechnol. 30, 460-465 (2012).
Chaudhuri, O. et al. Substrate stress relaxation regulates cell spreading. Nat. Commun. 6, 6365 (2015).
Zhao, X., Huebsch, N., Mooney, D. J. and Suo, Z. Stress-relaxation behavior in gels with ionic and covalent crosslinks. J. Appl. Phys. 107, 063509 (2010).
Tse, J. R. and Engler, A. J. Preparation of Hydrogel Substrates with Tunable Mechanical Properties. Curr. Protoc. Cell Biol. 47, 10.16.1-10.16.16 (2010).
Darnell, M. et al. Material microenvironmental properties couple to induce distinct transcriptional programs in mammalian stem cells. Proc. Natl. Acad. Sci. 115, E8368 (2018).
Wang, P.-Y. et al. Modulation of PEI-Mediated Gene Transfection through Controlling Cytoskeleton Organization and Nuclear Morphology via Nanogrooved Topographies. ACS Biomater. Sci. Eng. 3, 3283-3291 (2017).
Baslan, T. et al. Genome-wide copy number analysis of single cells. Nat. Protoc. 7, 1024-1041 (2012).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A hydrogel for CRISPR-Cas9 genome editing, wherein the hydrogel has a Young's modulus between about 2 kPa and about 200 kPa.

2. The hydrogel of claim 1, wherein the Young's modulus is selected from about 2 kPa, 2.5 kPa, 4 kPa, 10 kPa, 20 kPa, 30 kPa, 50 kPa, 100 kPa, and 150 kPa.

3. The hydrogel of claim 1, wherein the hydrogel is selected from a polyacrylamide (PAM) hydrogel, an alginate, a collagen, and polyethylene glycol (PEG).

4. The hydrogel of any one of claims 1 to 3, wherein the hydrogel comprises a PAM hydrogel.

5. The hydrogel of claim 4, wherein the PAM hydrogel comprises between about 2% wt % (w/v) to about 20% wt % (w/v) acrylamide and between about 0.010 wt % (w/v) to about 0.5 wt % (w/v) of bis-acrylamide.

6. The hydrogel of claim 5, wherein the PAM hydrogel further comprises one or more of ammonium persulfate, tetramethylethylenediamine (TEMED), phosphate-buffered saline (PBS), and water.

7. The hydrogel of claim 6, wherein the PAM hydrogel comprises a 1:100 total volume of ammonium sulfate and a 1:1000 total volume of tetramethylethylenediamine.

8. The hydrogel of claim 1, wherein the hydrogel comprises an alginate hydrogel.

9. The hydrogel of claim 8, wherein the alginate hydrogel is crosslinked with calcium ions.

10. The hydrogel of any one of claims 1 to 9, wherein the hydrogel further comprises one or more extracellular matrix (ECM) proteins bound to a surface thereof.

11. The hydrogel of claim 10, wherein the one or more ECM proteins comprise collagen I, laminin, RGD-containing peptides, and fibronectin.

12. The hydrogel of any one of claims 1 to 11, wherein the hydrogel comprises one or more seeded cells.

13. The hydrogel of claim 12, wherein the one or more cells is selected from U2OS.EGFP cells, human mesenchymal stem cells (MSCs), T cells, hematopoietic stem/progenitor cells (HSCs), neural stem/progenitor cells, human dermal fibroblasts, macrophages, and induced pluripotent stem cells (IPSCs).

14. The hydrogel of claim 12 or 13, wherein the one or more cells further comprise a Cas9 cargo.

15. The hydrogel of claim 14, wherein the Cas9 cargo is selected from a plasmid DNA, an mRNA, and a protein.

16. The hydrogel of claim 15, wherein the Cas9 cargo is selected from a mCherry-Cas9 plasmid, a Cas9 plasmid, a Cas9 mRNA, a Cas9 ribonucleoprotein (RNP), saCas9, dCas9, FokI-Fused dCas9, eSpCas9, xCas9, SpRY/SpG, HypaCas9, and High-Fidelity Cas9.

17. A method for preparing a hydrogel for CRISPR-Cas9 genome editing, the method comprising:
(a) providing a hydrogel having a Young's modulus between about 2 kPa and about 200 kPa;
(b) functionalizing the hydrogel with one or more extracellular matrix (ECM) proteins to form a functionalized hydrogel; and
(c) seeding the functionalized hydrogel with one or more cells.

18. The method of claim 17, further comprising incubating the one or more cells for a period of time.

19. The method of claim 18, further comprising transfecting the seeded cells with a Cas9 cargo.

20. The method of claim 19, wherein the transfecting comprises a transfection system selected from polyethylenimine (PEI), a lipid, a liposome, and electroporation.

21. The method of claim 20, wherein the lipid or liposome comprises one or more components selected from 2,3-dioleoyloxy-N-[2 (sperminecarboxamido)ethyl]-N,N-dimethyl-1-propaniminium trifluoroacetate (DOSPA), dioleoylphosphatidylethanolamine (DOPE), cholesterol, 2-dioleoyl-3-trimethylammonium-propane (DOTAP), 1,2-distearoyl-sn-glycero-3-phosphocholine (DSPC), 1,2-dioleoyl-sn-glycero-3-phosphocholine (DOPC), polyethylene glycol (PEG), and combinations thereof.

22. The method of claim 20, wherein the transfection system comprises linear or branched PEI.

23. The method of claim 20, wherein the transfection system comprises viral delivery.

24. The method of claim 23, wherein the viral delivery includes adeno-associated virus (AAV) or lentivirus vectors.

25. A method for improving DNA editing of CRISPR-Cas9, the method comprising:
(a) providing a hydrogel having a Young's modulus between about 2 kPa and about 200 kPa;
(b) seeding the hydrogel with one or more cells; and
(d) transfecting the seeded cells with a Cas9 cargo;
wherein DNA editing efficiency is enhanced.

26. The method of claim 25, wherein cytoskeletal alignment, cytoskeletal tension, or chromatin accessibility of the seeded cells is increased.

27. The method of claim 25, wherein homology-directed repair (HDR) efficiency is enhanced as compared to HDR efficiency of Cas9 cargo transfection of seeded cells on tissue culture plastic (TCP).

28. The method of claim 25, wherein the cells are selected from T-cells, human mesenchymal stem cells (MSCs), hematopoietic stem/progenitor cells (HSCs), neural stem/progenitor cells, human dermal fibroblasts, macrophages, and induced pluripotent stem cells (iPSCs).
